(19) United States
(12) Patent Application Publication
(10) Pub. No.: US 2024/0052372 A1
(43) Pub. Date: Feb. 15, 2024

(54) ALLELE-SPECIFIC GENOME EDITING OF THE NR2E3 MUTATION G56R

(71) Applicants: INSERM (INSTITUT NATIONAL DE LA SANTÉ ET DE LA RECHERCHE MÉDICALE), Paris (FR); UNIVERSITÉ DE MONTPELLIER, Montpellier (FR)

(72) Inventors: Vasiliki KALATZIS, Montpellier (FR); Michalista DIAKATOU, Montpellier (FR)

(21) Appl. No.: 18/278,494

(22) PCT Filed: Feb. 24, 2022
(86) PCT No.: PCT/EP2022/054629

§ 371 Date: Aug. 23, 2023

(30) Foreign Application Priority Data
Feb. 25, 2021 (EP) 21305224.4

(51) Int. Cl. C12N 15/90; C12N 9/22; C12N 15/11; A61K 38/46; A61K 31/7088; A61P 27/02

(52) U.S. Cl. CPC C12N 15/907; C12N 9/22; C12N 15/11; C12N 15/111; A61K 38/465; A61K 31/7088; A61P 27/02; C12N 2310/20; C12N 2320/34; C12N 2800/80

(57) ABSTRACT

Retinitis pigmentosa (RP) is an inherited retinal dystrophy that causes progressive vision loss. The second most common mutation causing autosomal dominant (ad) RP is the G56R mutation in NR2E3, a transcription factor essential for photoreceptor development. The G56R variant is exclusively responsible for all cases of NR2E3-associated adRP. Currently, there is no treatment for NR2E3-related, or other, adRP, but genome editing holds promise. In this study, the inventors developed a CRISPR/Cas strategy to specifically knockout the mutant G56R allele of NR2E3 and performed a proof-of-concept study in iPSC of an adRP patient. They demonstrate allele-specific knockout of the mutant G56R allele in the absence of off-target events. Furthermore, they validated this knockout strategy in an exogenous overexpression system. They showed for the first time that G56R iPSC, as well as G56R-CRISPR iPSC, can differentiate into NR2E3-expressing retinal organoids. Overall, they demonstrate that G56R allele-specific knockout by CRISPR/Cas could be a clinically relevant approach to treat NR2E3-associated adRP.Thus, the invention refers to a site-directed genetic engineering system for specifically editing an allele containing c.166G>A mutation in NR2E3 in the genome of an individual and its use for treating autosomal dominant retinitis pigmentosa.

### FIELD OF THE INVENTION

The invention relates to the field of therapeutic treatment by genome editing. In particular, the invention relates to a site-directed genetic engineering system for specifically silencing an allele containing the c.166G>A mutation in NR2E3 in the genome of a subject in need thereof, and its use in gene therapy and/or cell therapy, in more particular to treat retinitis pigmentosa

### BACKGROUND OF THE INVENTION

Inherited retinal dystrophies (IRD) are a group of disorders characterized by progressive vision loss. This is due to degeneration of the light-sensing cells of the retina, the photoreceptors. The most common IRD form is rod-cone dystrophy, more commonly known as Retinitis Pigmentosa (RP), which affects ˜1/4000 people worldwide [1]. RP is characterized by tunnel vision due to the initial degeneration of rod, followed by cone, photoreceptors [2]. RP is caused by mutations in over 80 genes and can be transmitted in autosomal dominant (namely one mutant allele is sufficient for disease manifestation), autosomal recessive (namely two mutant alleles are needed for disease manifestation) and X-linked (mutation in X-chromosome) inheritance patterns [1]. The most common mutation responsible for autosomal dominant retinitis pigmentosa (adRP) is the missense mutation c.68C>A (p.Pro23H) in RHO, the gene encoding rhodopsin, the visual pigment of rod photoreceptors. The second most common mutation for adRP is the c.166G>A (p.Gly56Arg; G56R) mutation in the gene NR2E3. The G56R mutation is exclusively responsible for all NR2E3-associated adRP cases, and accounts for 1-2% of total adRP cases in America and 3.5% in Europe [4-6].

NR2E3 (Nuclear Receptor subfamily 2 group E member 3) is a photoreceptor-specific transcription factor and key player in the development and maintenance of rod photoreceptors [7-9]. More specifically, NR2E3 acts in a complex with CRX, NRL and NR1D1 to promote the transcription of rod genes, such as RHO, while repressing the transcription of cone genes [10,11]. NR2E3 has a typical nuclear receptor structure with two main domains, a DNA Binding Domain (DBD) and a Ligand Binding Domain (LBD) [12]. In addition to DNA binding, the DBD is responsible for the interaction of NR2E3 with CRX [9]. By contrast, the LBD mediates the formation of NR2E3 homodimers, which are necessary for transcriptional repression [13,14]. The highly conserved G56 residue is localized within the DBD of NR2E3. The disease mechanism of the G56R variant remains elusive. Some hypotheses are that the G56R mutation reduces DNA binding and subsequent RHO activation [15-17], weakens CRX binding or decreases homodimerization of NR2E3.

Currently, there is no treatment for RP, including NR2E3-associated adRP. However, the advent of the powerful CRISPR/Cas technology provides hope for genome editing as a gene therapy approach [18-20].

The CRISPR/Cas system comprises two elements, a Cas endonuclease and a 20-nt guide RNA (gRNA). The gRNA is situated next to a 3-nt sequence known as a protospacer adjacent motif (PAM) [18]. The most commonly used Cas is Cas9 from Streptococcus pyogenes (Sp), which recognizes an NGG PAM sequence. The combination of the PAM and gRNA molecule guides the Cas9 to the target sequence in the host DNA where it induces a double-strand break (DSB). The DSB is then repaired by the cell machinery by one of two main pathways [21]. The first pathway is homologous-directed repair (HDR), which takes place during the S/G2 phase of dividing cells [22]. HDR is exploited for gene correction by providing a DNA sequence repair template along with the CRISPR/Cas system [23]. The second pathway is non-homologous end joining (NHEJ) that is recruited during all phases of the cell cycle in the absence of a repair template.

In most cases where genome-editing is envisaged, the ideal choice would be to correct a mutant allele by the HDR pathway. However, this can be challenging in post-mitotic cells, such as photoreceptors. By contrast, knocking out a mutant allele could be efficient and clinically relevant, especially for an autosomal dominant disorder. This approach takes advantage of the susceptibility to error of the NHEJ pathway, namely the introduction of small insertions or deletions (indels) at the cleavage site. These indels may lead to a frameshift and a premature termination codon (PTC) [24]. Depending on where the premature stop codon emerges, the mRNA may be subjected to nonsense-mediated decay (NMD). A risk of mutant allele ablation is haploinsufficiency, i.e. the insufficiency of a single copy of the target gene to produce adequate protein levels for cell function. In the case of NR2E3, however, this is likely not an issue. Homozygous mutations in NR2E3 give rise to an autosomal recessive IRD called Enhanced S Cone Syndrome (ESCS) and one of the most common is a loss-of-function splice site mutation in intron 1 that spares only the first exon [25]. As is common for an autosomal recessive IRDs, heterozygous carriers are healthy subjects, as are heterozygous littermates of the Nr2e3^{-/-} mouse models [26,27]. Taken together, these data suggest that one wild type (WT) NR2E3 allele is sufficient for the correct development and function of rod photoreceptors.

Herein, the inventors designed a CRISPR/Cas9 genome-editing strategy to specifically ablate the mutant G56R allele in induced pluripotent stem cells (iPSCs) of an NR2E3-adRP patient.

### SUMMARY OF THE INVENTION

The invention relates to a site-directed genetic engineering system for specifically editing an allele containing the c.166G>A mutation in NR2E3 in the genome of a subject in need thereof, comprising:
(i) at least one guide nucleic acid comprising the nucleic acid sequence selected from the group consisting of SEQ ID NO: 1, SEQ ID NO: 2, and SEQ ID NO: 3 and
(ii) at least one Clustered regularly interspaced short palindromic repeats (CRISPR) associated nuclease.

In particular, the present invention is defined by the claims.

### DETAILED DESCRIPTION OF THE INVENTION

Retinitis pigmentosa is an inherited retinal dystrophy causing visual impairment. The second most common mutation of the autosomal dominant form of the disease is the G56R mutation in NR2E3. NR2E3 is a transcription factor essential for rod differentiation. There is no current treatment for this disease. Genome editing offers a host of therapeutic options for IRDS in terms of gene and cell therapy but the feasibility of clinical translation may be variable. For example, gene correction requires HDR and thus may not reach therapeutic efficiency for gene therapy, due to the post-mitotic nature of photoreceptors, but holds promise for cell therapy. By contrast, a HDR-independent strategy involving specific knockout of a mutant allele could be a potentially efficient gene therapy approach. This would be particularly pertinent in the case of G56R, as this mutation exclusively causes all NR2E3-associated adRP forms. Therefore, a single therapeutic product could potentially treat all patients. In this study, the inventors developed a CRISPR strategy to specifically knock out the mutant G56R allele in patient derived iPSCs. They validated this approach in HEK293 cells where a truncated and mislocalized CRISPRed protein was detected. Furthermore, differentiation of iPSCs to retinal organoids revealed that the NR2E3 CRISPRed iPSCs are able to differentiate into photoreceptor cells that express mature markers such as rhodopsin and RG Opsin. To conclude, G56R allele specific knock out could be a clinically appealing approach to treat NR2E3 related autosomal dominant retinitis pigmentosa.

Main Definition

As used herein, the term “guide acid nucleic” also known as “gRNA” generally refers to an RNA molecule (or a group of RNA molecules collectively) that can bind to a CRISPR protein and target the CRISPR protein to a specific location within a target DNA. A guide RNA can comprise two segments: a DNA-targeting guide segment and a protein-binding segment. The DNA-targeting segment comprises a nucleotide sequence that is complementary to (or at least can hybridize to under stringent conditions) a target sequence. The protein-binding segment interacts with a CRISPR protein, such as a Cas9 or Cas9-related polypeptide. These two segments can be located in the same RNA molecule or in two or more separate RNA molecules. When the two segments are in separate RNA molecules, the molecule comprising the DNA-targeting guide segment is referred to as the CRISPR RNA (“crRNA”), while the molecule comprising the protein-binding segment is referred to as the trans-activating RNA (“tracrRNA”). Typically the crRNA comprises at least one spacer sequence and at least one repeat sequence, or a portion thereof, linked to the 5′ end of the spacer sequence. The design of a crRNA of this invention will vary based on the CRISPR-Cas system in which the crRNA is to be used. The crRNAs of this invention are synthetic, made by man and not found in nature. Typically, a crRNA may comprise, from 5′ to 3′, a repeat sequence (full length or portion thereof (“handle”)), a spacer sequence, and a repeat sequence (full length or portion thereof). In some embodiments, a crRNA may comprise, from 5′ to 3′, a repeat sequence (full length or portion thereof (“handle”)) and a spacer sequence. The tracr nucleic acid comprises from 5′ to 3′ a bulge, a nexus hairpin and terminal hairpins, and optionally, at the 5′ end, an upper stem (See, Briner et al. (2014) Molecular Cell. 56(2):333-339). A tracrRNA functions in hybridizing to the repeat portion of mature or immature crRNAs, recruits Cas9 protein to the target site, and may facilitate the catalytic activity of Cas9 by inducting structural rearrangement. Sequences for tracrRNAs are specific to the CRISPR-Cas Type II system and can be variable. When a phasmid is engineered to comprise a heterologous Type II CRISPR-Cas system in addition to a Type II crRNA, any tracr nucleic acid, known or later identified, can be used. In some embodiments, the tracr nucleic acid is fused to the crRNA of the invention to farm a single guide nucleic acid.

As used herein, the term “CRISPR associated nuclease” has its general meaning in the art and refers to segments of prokaryotic DNA containing clustered regularly interspaced short palindromic repeats (CRISPR) and associated nucleases, especially associated nucleases encoded by Cas genes. In bacteria the CRISPR/Cas loci encode RNA-guided adaptive immune systems against mobile genetic elements (viruses, transposable elements and conjugative plasmids). Three types of CRISPR systems have been identified. CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA). The CRISPR/Cas nucleases Cas9 and Cpf1 belong to the type II and type V CRISPR/Cas system and have strong endonuclease activity to cut target DNA. Cas9 is guided by a mature crRNA that contains about 20 nucleotides of unique target sequence (called spacer) and a trans-activating small RNA (tracrRNA) that also serves as a guide for ribonuclease III-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG for S. Pyogenes Cas9) protospacer adjacent motif (PAM) to specify the cut site (the 3^{rd} or the 4^{th} nucleotide upstream from PAM).

As used herein, the term “Cas9” or “Cas9 nuclease” refers to an RNA-guided nuclease comprising a Cas9 protein, or a fragment thereof (e.g., a protein comprising an active or inactive DNA cleavage domain of Cas9, and/or the gRNA binding domain of Cas9). A Cas9 nuclease is also referred to sometimes as a casn1 nuclease or a CRISPR (clustered regularly interspaced short palindromic repeat)-associated nuclease. CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (mc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821(2012), the entire contents of which is hereby incorporated by reference. Cas9 recognizes a short motif in the CRISPR repeat sequences (the PAM or protospacer adjacent motif) to help distinguish self from non-self. Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, the term “Cas9” refers to Cas9 from: Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisI (NCBI Ref: NC_018721.1); Streptococcus thermophilus (NCBI Ref: YP_820832.1); Listeria innocua (NCBI Ref: NP_ 472073.1); Campylobacter jejuni (NCBI Ref: YP_002344900.1); or Neisseria. meningitidis (NCBI Ref: YP_002342100.1).

As used herein, the term “double strand break” or “DSB” refers to two breaks in a nucleic acid molecule, e.g., a DNA molecule: a first break in a first strand of the nucleic acid molecule, and a second break in a second strand of the nucleic acid molecule

As used herein, the term “recombination” has its general meaning in the art and refers to pathways that repairs double strand break.

As used herein, the term “non-homologous end joining (NHEJ) recombination” has its general meaning in the art and refers to a predominant DSB-repair mechanism in mammalian cells, throughout the cell cycle, including during S and G2 phases. NHEJ occurs via three main steps: (1) DSB recognition, (2) processing of nonligatable DNA termini, and (3) joining of two suitable DSBs. Noteworthy here, NHEJ can also directly religate the broken DNA ends and does not require DNA end resection for repair initiation. Classical NHEJ (c-NHEJ) is mediated by the Ku70/Ku80 heterodimer which binds to DSBs within seconds and dictates NHEJ pathway choice. [44]

As used herein, the term “NR2E3” also known as “Nuclear Receptor subfamily 2 Group E Member 3” or “photoreceptor cell-specific nuclear receptor (PNR)” has its general meaning in the art and refers to a photoreceptor-specific transcription factor. The role of NR2E3 in the retina is well-established; it is responsible for the differentiation and maintenance of rod photoreceptors [9-11]. NR2E3 acts in a complex with CRX, NRL, NR1D1 and possibly more factors to promote the transcription of rod genes, such as rhodopsin, while inhibiting the transcription of cone genes, such as S, M and L Opsins. NR2E3 has two main domains, a DNA Binding Domain (DBD) and a Ligand Binding Domain (LBD). The NR2E3 gene is an autosomal dominant gene located in chromosome 15. Its Entrez reference is 10002. Mutations in human NR2E3 are associated with several forms of retinal degeneration that vary in phenotype and were categorized by their clinical diagnosis as they were discovered.

As used herein, the term “c.166G>A mutation in NR2E3”, also known as “G56R”, is one of the most common mutation responsible for inherited retinal dystrophies (IRD), and in particular for autosomal dominant retinitis pigmentosa (adRP). The G56 residue is localized within the DNA binding domain and it is a highly conserved residue. The effect of the G56R mutation remains elusive. The c.166G>A mutation is qualified as heterozygous if the mutation is different from one allele to the other. In other word, a heterozygous c.166G>A mutation in the NR2E3 gene means that one allele contains the c.166G>A mutation, while the other allele does not.

As used herein, the term “retinitis pigmentosa” (RP), also known as “rod-cone dystrophy”, has its general meaning in the art and refers to a genetic disorder causing loss of vision, which affects 1/4000 people worldwide. Retinitis pigmentosa is characterized by trouble seeing at night and decreased peripheral vision (side vision) and by tunnel vision due to the initial degeneration of rod, followed by cone, photoreceptors [1]. Retinitis pigmentosa (RP) thus is also known as “rod-cone dystrophy” and is one of the most common forms of inherited retinal degeneration. There are multiple genes that, when mutated, can cause the retinitis pigmentosa phenotype. Inheritance patterns of RP have been identified as autosomal dominant (one mutant allele is sufficient for disease manifestation), autosomal recessive (two mutant alleles are needed for disease manifestation), X-linked (mutation in X-chromosome), and maternally (mitochondrially) acquired.

As used herein, the term “vector” has its general meaning in the art and refers to the vehicle by a nucleic acid molecule can be introduced into a host cell, so as to transform the host and promote expression (e.g. transcription and translation) of the introduced sequence. The terms “Gene transfer” or “gene delivery” refer to methods or systems for reliably inserting foreign DNA into host cells. Such methods can result in transient expression of non-integrated transferred DNA, extrachromosomal replication and expression of transferred replicons (e.g. episomes), or integration of transferred genetic material into the genomic DNA of host cells. Cells could be hematopoietic stem cells (e.g. CD34+ cell fraction) or hematopoietic progenitor cells (particularly monocytic progenitors or microglia precursors) isolated from the bone marrow or the blood of the patient (autologous) or from a donor (allogeneic) genetically modified to stably express APPsa or a fragment derived from it by transduction with a vector, particularly a lentiviral vector expressing APPsa under the control of a non-specific (e.g.: phosphoglycerate kinase, EF1alpha) or specific (monocytic-macrophage or microglia specific e.g. CD68 or CD11b) native or modified promoter. According to the invention, vectors include viral vectors or non-viral vectors. Non-viral vectors mainly comprise chemical systems that are not of viral origin and generally include chemical methods such as cationic liposomes and polymers. Efficiency of these vectors may sometimes be less than viral systems in gene transduction, but their cost-effectiveness, availability, and more importantly less induction of immune system and no limitation in size of transgenic DNA compared with viral systems have made them more effective for gene delivery.

Viral vectors useful in the practice of the present invention can be constructed utilizing methodologies well known in the art of molecular biology. Typically, viral vectors carrying transgenes are assembled from polynucleotides encoding the transgene, suitable regulatory elements and elements necessary for production of viral proteins which mediate cell transduction. Examples of viral vector include but are not limited to retrovirus, adenovirus, adeno-associated virus (AAV), herpes virus, pox virus, human foamy virus (HFV), and lentivirus. All viral vector genomes have been modified by deleting some areas of their genomes so that their replication becomes deranged and it makes them safer to administrate to a patient. During the past few years, some viral vectors with specific receptors have been designed that could transfer the transgenes to some other specific cells, which are not their natural target cells (retargeting).

As used herein, the term “AAV vector” refers to a vector derived from an adeno-associated virus serotype, including without limitation AAV1, AAV2, AAV3, AAV4, AA5, AAV6, AAV7, AAV8, AAV9, AAVrh10 or any other serotypes of AAV that can infect humans, monkeys or other species. AAV vectors can have one or more of the AAV wild-type genes deleted in whole or part, preferably the rep and/or cap genes, but retain functional flanking ITR sequences. Functional ITR sequences are necessary for the rescue, replication and packaging of the AAV virion. Thus, an AAV vector is defined herein to include at least those sequences required in cis for replication and packaging (e.g., functional ITRs) of the virus. The ITRs need not be the wild-type nucleotide sequences, and may be altered, e. g by the insertion, deletion or substitution of nucleotides, so long as the sequences provide for functional rescue, replication and packaging. AAV expression vectors are constructed using known techniques to at least provide as operatively linked components in the direction of transcription, control elements including a transcriptional initiation region, the nucleic acid molecule of the present invention and a transcriptional termination region. The control elements are selected to be functional in a mammalian cell. The resulting construct which contains the operatively linked components is bounded (5′ and 3′) with functional AAV ITR sequences. By “adeno-associated virus inverted terminal repeats ” or “AAV ITRs” is meant the art-recognized regions found at each end of the AAV genome which function together in cis as origins of DNA replication and as packaging signals for the virus. AAV ITRs, together with the AAV rep coding region, provide for the efficient excision and rescue from, and integration of a nucleotide sequence interposed between two flanking ITRs into a mammalian cell genome. The nucleotide sequences of AAV ITR regions are known. See, e.g., Kotin, 1994; Berns, KI “Parvoviridae and their Replication” in Fundamental Virology, 2nd Edition, (B. N. Fields and D. M. Knipe, eds.) for the AAV-2 sequence. As used herein, an “AAV ITR” does not necessarily comprise the wild-type nucleotide sequence, but may be altered, e.g., by the insertion, deletion or substitution of nucleotides. Additionally, the AAV ITR may be derived from any of several AAV serotypes, including without limitation, AAV1, AAV2, AAV3, AAV4, AAV5, AAV6, etc. Furthermore, 5′ and 3′ ITRs which flank a selected nucleotide sequence in an AAV vector need not necessarily be identical or derived from the same AAV serotype or isolate, so long as they function as intended, i.e., to allow for excision and rescue of the sequence of interest from a host cell genome or vector, and to allow integration of the heterologous sequence into the recipient cell genome when AAV Rep gene products are present in the cell. Additionally, AAV ITRs may be derived from any of several AAV serotypes, including without limitation, AAV1, AAV2, AAV3, AAV4, AAV5, AAV6, etc. Furthermore, 5′ and 3′ ITRs which flank a selected nucleotide sequence in an AAV expression vector need not necessarily be identical or derived from the same AAV serotype or isolate, so long as they function as intended, i.e., to allow for excision and rescue of the sequence of interest from a host cell genome or vector, and to allow integration of the DNA molecule into the recipient cell genome when AAV Rep gene products are present in the cell. In some embodiments, the AAV vector of the present invention is selected from vectors derived from AAV serotypes having tropism for and high transduction efficiencies in cells of the mammalian central and peripheral nervous system, particularly neurons, neuronal progenitors, astrocytes, oligodendrocytes and glial cells. In some embodiments, the AAV vector is an AAV4, AAV9 or an AAVrh10 that have been described to well transduce brain cells especially neurons. In some embodiments, the AAV vector of the present invention is a double-stranded, self-complementary AAV (scAAV) vector. Alternatively to the use of single-stranded AAV vector, self-complementary vectors can be used. The efficiency of AAV vector in terms of the number of genome-containing particles required for transduction, is hindered by the need to convert the single-stranded DNA (ssDNA) genome into double-stranded DNA (dsDNA) prior to expression. This step can be circumvented through the use of self-complementary vectors, which package an inverted repeat genome that can fold into dsDNA without the requirement for DNA synthesis or base-pairing between multiple vector genomes. Resulting self-complementary AAV (scAAV) vectors have increased resulting expression of the transgene. For an overview of AAV biology, ITR function, and scAAV constructs, see McCarty D M. Self-complementary AAV vectors; advances and applications. Mol. Ther. 2008 October; 16 (10): at pages 1648-51, first full paragraph, incorporated herein by reference for disclosure of AAV and scAAV constructs, ITR function, and role of ΔTRS ITR in scAAV constructs. A rAAV vector comprising a ΔTRS ITR cannot correctly be nicked during the replication cycle and, accordingly, produces a self-complementary, double-stranded AAV (scAAV) genome, which can efficiently be packaged into infectious AAV particles. Various rAAV, ssAAV, and scAAV vectors, as well as the advantages and drawbacks of each class of vector for specific applications and methods of using such vectors in gene transfer applications are well known to those of skill in the art (see, for example, Choi V W, Samulski R J, McCarty D M. Effects of adeno-associated virus DNA hairpin structure on recombination. J. Virol. 2005 June; 79(11):6801-7; McCarty D M, Young S M Jr, Samulski R J. Integration of adeno-associated virus (AAV) and recombinant AAV vectors. Annu Rev Genet. 2004; 38:819-45; McCarty D M, Monahan P E, Samulski R J. Self-complementary recombinant adeno-associated virus (scAAV) vectors promote efficient transduction independently of DNA synthesis. Gene Ther. 2001 August; 8(16):1248-54; and McCarty D M. Self-complementary AAV vectors; advances and applications. Mol. Ther. 2008 October; 16(10):1648-56; all references cited in this application are incorporated herein by reference for disclosure of AAV, rAAV, and scAAV vectors).

As used herein, the term “subject” or “patient” refers to any mammals, such as a rodent, a feline, a canine, and a primate. Particularly, in the present invention, the subject is a human afflicted with retinitis pigmentosa, in particular with autosomal dominant retinitis pigmentosa, and more particularly of NR2E3-associated autosomal dominant retinitis pigmentosa.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment.

As used herein, the term “therapeutically effective amount” refers to an amount sufficient to produce the desired physiological effect or amount capable of achieving the desired result, particularly for treating the disorder or disease condition, including reducing or eliminating one or more symptoms or manifestations of the disorder or disease. The effective amount will vary with the age, general condition of the subject, the severity of the condition being treated, the nature of any concurrent treatment, the pharmaceutically acceptable carrier used, and like factors within the knowledge and expertise of those skilled in the art. As appropriate, an “effective amount” in any individual case can be determined by one of skill in the art by reference to the pertinent texts and literature and/or by using routine experimentation.

As used herein, the term “pharmaceutical composition” refers to a composition described herein, or pharmaceutically acceptable salts thereof, with other agents such as carriers and/or excipients. The pharmaceutical compositions as provided herewith typically include a pharmaceutically acceptable carrier.

As used herein, the term “pharmaceutically” or “pharmaceutically acceptable” refers to medium and compositions that do not produce an adverse, allergic or other untoward reaction when administered to a mammal, especially a human, as appropriate. A pharmaceutically acceptable medium comprises any of standard pharmaceutically accepted mediums known to those of ordinary skill in the art, in particular in formulating pharmaceutical compositions to be administered to the eye.

As used herein, the term “induced pluripotent stem cells” (iPSCs) has its general meaning in the art and refers to a type of pluripotent stem cells that can be generated directly from a somatic cell. Pluripotent stem cells hold promise in the field of regenerative medicine. iPSCs are genetically reprogrammed adult cells that exhibit a pluripotent stem cell-like state similar to embryonic stem cells (ESCs). They are artificially generated stem cells that are not known to exist in the human body but show qualities similar to those of ESC. Generating such cells is well known in the art as discussed in Ying WANG et al. (47) as well as in Lapillonne H. et al. (48) and in J. DIAS et al. (49). iPSCs are typically derived by introducing products of specific sets of pluripotency-associated genes, or “reprogramming factors”, into a given cell type, which are well known to one skilled in the art. For instance, iPSCs may be generated from human fibroblasts. The generation of iPSCs is crucially dependent on the transcription factors used for the induction. Since iPSCs can be derived directly from adult tissues, they not only bypass the need for embryos, but can be made in a patient-matched manner, which means that each individual could have their own pluripotent stem cell line.

As used herein the term “photoreceptor cells” has its general meaning in the art and refers to are light-sensitive ocular cells. There are currently three known types of photoreceptor cells in mammalian eyes: rods, cones, and intrinsically photosensitive retinal ganglion cells. The two classic photoreceptor cells are rods and cones, each contributing information used by the visual system to form a representation of the visual world, sight. The rods are narrower than the cones and distributed differently across the retina, but the chemical process in each that supports phototransduction is similar.

As used herein, the term “retinal progenitor cells” refers to cells that differentiate into the various cell types of the retina during development. In the vertebrate, these retinal cells differentiate into seven cell types, including retinal ganglion cells, amacrine cells, bipolar cells, horizontal cells, rod photoreceptors, cone photoreceptors, and Muller glia cells.

As used herein, the term “gene therapy” has its general meaning in the art and refers to delivering nucleic acids into a patient's cells as a drug to treat disease. Gene therapy includes several approaches such as replacing a mutated gene that causes a medical problem with a healthy copy of the gene, inactivating (or “knocking-out”) the mutated gene, and introducing a new gene to help the body to fight or treat disease.

As used herein, the term “cell therapy” has its general meaning in the art and refers to transplanting cells in order to restore tissue or organ function.

As used herein, the term “regenerative medicine” has its general meaning in the art and refers to a process of replacing, engineering or regenerating human or animal cells, tissues or organs to restore or establish normal function.

A Site-Directed Genetic Engineering System for Specifically Editing an Allele Containing the c.166G>A Mutation in NR2E3 in the Genome, and its use for Treating Retinitis Pigmentosa.

Accordingly, in a first aspect, the invention refers to a site-directed genetic engineering system for specifically editing an allele containing the c.166G>A mutation in NR2E3 in the genome of a subject in need thereof.

In particular the invention refers to a site-directed genetic engineering system for specifically editing an allele containing the c.166G>A mutation in NR2E3 in the genome of a subject in need thereof, comprising:
(i) at least one guide nucleic acid comprising the nucleic acid sequence selected from the group consisting of SEQ ID NO: 1, SEQ ID NO: 2, and SEQ ID NO: 3 and
(ii) at least one Clustered regularly interspaced short palindromic repeats (CRISPR) associated nuclease.

The inventors designed gRNA which are able to specifically recognize the mutant allele containing the c.166G>A mutation in NR2E3 gene. Indeed, the gRNA designed by the inventors are able to target the mutant allele containing the c.166G>A mutation in NR2E3 gene while not targeting the wild type allele, namely the allele without c.166G>A mutation in NR2E3 gene. Indeed, silencing the G56R mutant allele of NR2E3 will allow the expression of WT NR2E3 in the absence of a mutant protein, this expression level should be sufficient for normal retinal development.

Thus, in particular the invention refers to a site-directed genetic engineering system for specifically silencing an allele containing the c.166G>A mutation in NR2E3 in the genome of a subject in need thereof, comprising:
(i) at least one guide nucleic acid comprising the nucleic acid sequence selected from the group consisting of SEQ ID NO: 1, SEQ ID NO: 2, and SEQ ID NO: 3 and
(ii) at least one Clustered regularly interspaced short palindromic repeats (CRISPR) associated nuclease.

Thus, in some embodiment, the guide nucleic acid consist of the nucleic acid sequence SEQ ID NO: 1, SEQ ID NO: 2 or SEQ ID NO: 3.

TABLE 1
Sequence of gRNA

| Guide | | |
|---|---|---|
| nucleic | | |
| acid | SEQ ID NO: | Sequence |
| gRNA1 | SEQ ID NO: 1 | ^{5′}AGCAGCAGCAGGAAGCACTA^{3′} |
| gRNA2 | SEQ ID NO: 2 | ^{5′}GTGCGGAGACAGCAGCAGCA^{3′} |
| gRNA1′ | SEQ ID NO: 3 | ^{5′}GAGCAGCAGCAGGAAGCACTA^{3′} |

In some embodiment, the CRISPR associated nuclease is a CRISPR/Cas nuclease.

Various CRISPR/Cas nucleases can be used in this invention. Non-limiting examples of suitable CRISPR/CRISPR/Cas nucleases include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966. See e.g., WO2014144761 WO2014144592, WO2013176772, US20140273226, and US20140273233, the contents of which are incorporated herein by reference in their entireties

In some embodiment, the CRISPR/Cas nuclease is derived from a Cas9 protein. The Cas9 protein can be from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina, inter alia.

In some embodiments, the Cas9 nuclease can have a nucleotide sequence identical to the wild type Streptococcus pyogenes sequence.

In some embodiment, the guide nucleic acid comprise or consist of the nucleic acid sequence selected from the group consisting of SEQ ID NO: 1 or SEQ ID NO: 3, and the CRISPR-associated nuclease is wild type Cas9.

In some embodiments, the CRISPR-associated nuclease can be a sequence from other species, for example other Streptococcus species, such as thermophilus; Pseudomonas aeruginosa, Escherichia coli, or other sequenced bacteria genomes and archaea, or other prokaryotic microorganisms. Alternatively, the wild type Streptococcus pyogenes Cas9 sequence can be modified. Alternatively, the Cas9 nuclease sequence can be for example, the sequence contained within a commercially available vector such as pX330, pX260 or pMJ920 from Addgene (Cambridge, MA). In some embodiments, the Cas9 endonuclease can have an amino acid sequence that is a variant or a fragment of any of the Cas9 endonuclease sequences of Genbank accession numbers KM099231.1 GL669193757; KM099232.1; GL669193761; or KM099233.1 GL669193765 or Cas9 amino acid sequence of pX330, pX260 or pMJ920 (Addgene, Cambridge, MA).

In some embodiment, the CRISPR/Cas nuclease is a high efficiency CRISPR associated protein 9 (eSpCas9 (1.1)), as described in Slaymaker et al. [31].

In some embodiment, the guide nucleic acid comprise or consist of the nucleic acid sequence SEQ ID NO: 1 or SEQ ID NO: 3, and the CRISPR/Cas nuclease is a high efficiency CRISPR-associated protein 9 (eSpCas9 (1.1)).

In some embodiment, the CRISPR/Cas nuclease is an engineered variant of SpCas9, such as VQR CRISPR/Cas9 (SpCas9-VQR).

This variants are created to increase the repertoire of genomic target sequences. They are well known in the art as discussed in Kleinstiver et al. [32]

In some embodiment, the CRISPR/Cas nuclease is SpCas9-VQR.

In some embodiment, the guide nucleic acid comprise or consist of the nucleic acid sequence SEQ ID NO: 2, and the CRISPR/Cas nuclease is an engineered VQR CRISPR/Cas9.

In some embodiment, the elements (i) and (ii) of the system according to the invention may be contained in at least one vector.

Thus, in particular the invention refers to a site-directed genetic engineering system for specifically silencing an allele containing the c.166G>A mutation in NR2E3 in the genome of a subject in need thereof, comprising at least one vector comprising:
(i) at least one guide nucleic acid comprising the nucleic acid sequence selected from the group consisting of SEQ ID NO: 1, SEQ ID NO: 2 and SEQ ID NO: 3; and
(ii) at least one Clustered regularly interspaced short palindromic repeats (CRISPR) associated nuclease.

In some embodiments, more than one vector comprising the elements (i), and/or (ii) will be used. These vectors may be identical or different.

In some embodiments, at least one vector comprises the elements of (i) and at least one vector comprises the elements of (ii).

Thus, in particular the invention refers to a site-directed genetic engineering system for specifically silencing an allele containing the c.166G>A mutation in NR2E3 in the genome of a subject in need thereof, comprising:
(iii) at least one vector comprising at least one guide nucleic acid comprising the nucleic acid sequence selected from the group consisting of SEQ ID NO: 1, SEQ ID NO: 2 and SEQ ID NO: 3; and
(iv) at least one vector comprising at least one Clustered regularly interspaced short palindromic repeats (CRISPR) associated nuclease.

In some embodiment, the vectors are viral vectors or non-viral vectors.

Viral and non-viral vectors that may be used according to the invention are well known to the skilled in the art, and are, for example, described in Nayerossadat et al. [45].

Viral vectors are successful gene therapy systems such as retrovirus, adenovirus (types 2 and 5), adeno-associated virus (AAV), herpes virus, pox virus, human foamy virus (HFV), and lentivirus. All viral vector genomes have been modified by deleting some areas of their genomes so that their replication becomes deranged and it makes them safer to administrate to a patient. During the past few years, some viral vectors with specific receptors have been designed that could transfer the transgenes to some other specific cells, which are not their natural target cells (retargeting).

In some embodiment, the viral vectors are selected the group consisting of retroviral vectors, adenoviral vectors, adeno-associated virus vectors, herpes simplex virus vectors, lentivectors, poxvirus vectors and Epstein-Barr virus vectors, and in particular is selected from adeno-associated virus vectors.

In some embodiment, the viral vectors are adeno-associated virus vectors.

In some embodiment, the subject is a human afflicted with retinitis pigmentosa, in particular with autosomal dominant retinitis pigmentosa, and more particularly with NR2E3-associated autosomal dominant retinitis pigmentosa.

In some embodiment, the subject have an allele containing the c.166G>A mutation in NR2E3 gene. In some embodiment, the subject have only an allele containing the c.166G>A mutation in NR2E3 gene. In other words, in some embodiment, the subject have a heterozygous c.166G>A mutation in the NR2E3 gene.

Treatment based on the administration of a system according to the invention and described herein may be used in gene therapy for treating a large number of subject afflicted with retinitis pigmentosa, in particular with autosomal dominant retinitis pigmentosa, and more particularly with NR2E3-associated autosomal dominant retinitis pigmentosa, for which there is currently no treatment available.

Indeed, the expression of the system according to the invention allows the binding of the CRISPR protein to a locus cognate to the gRNA and in vivo generation of a double strand break (DSB), and wherein the in vivo recombination of said DSB results in silencing the allele containing the c.166G>A mutation in NR2E3 gene.

Accordingly, in a second aspect, the invention refers to a site-directed genetic engineering system according to the invention and described herein for use in gene therapy.

Accordingly, in particular, the invention refers to a site-directed genetic engineering system according to the invention and described herein for use in the treatment of retinitis pigmentosa in a subject in need thereof.

In other word, the invention refers to a method for treating retinitis pigmentosa in a subject in need thereof comprising administering to the subject a therapeutically effective amount of the site-directed genetic engineering system according to the invention.

In particular, the invention refers to a method for treating retinitis pigmentosa in a subject in need thereof comprising administering to the subject a therapeutically effective amount of the site-directed genetic engineering system according to the invention, wherein the CRISPR protein binds to a locus cognate to the gRNA and generate a double strand break (DSB), and wherein the in vivo recombination of said DSB results in silencing the c.166G>A mutation.

In some embodiment, the recombination of said DSB is non-homologous end joining (NHEJ) recombination

In some embodiment, the site-directed genetic engineering system according to the invention is administered in combination with a vector comprising the unmutated NR2E3 gene (Genbank NM_014249 or NM_016346).

In some embodiment, the retinitis pigmentosa is autosomal dominant retinitis pigmentosa, and more particularly NR2E3-associated autosomal dominant retinitis pigmentosa.

In some embodiment, the subject have a heterozygous c.166G>A mutation in the NR2E3 gene.

In some embodiment, the elements of (i), (ii) of the system according to the invention may be administered to the individual to be treated through other means, without the need for a vector.

When the system according to the invention is used in the treatment of retinitis pigmentosa, it may in particular be administrated in the form of a pharmaceutical composition further comprising a pharmaceutically acceptable medium.

Thus, the invention also refers to as pharmaceutical composition comprising the site-directed genetic engineering system according to the invention.

In a particular embodiment, the pharmaceutical composition is suitable for a local administration to the individual to be treated, such as is suitable for an administration to the eye of the individual to be treated.

Ocular drug delivery has been a major challenge to pharmacologists and drug delivery scientists due to its unique anatomy and physiology. Static barriers (different layers of cornea, sclera, and retina including blood aqueous and blood-retinal barriers), dynamic barriers (choroidal and conjunctival blood flow, lymphatic clearance, and tear dilution), and efflux pumps in conjunction pose a significant challenge for delivery of a drug alone or in a dosage form, especially to the posterior segment.

The three primary methods of delivery of pharmaceutical compositions to the eye are topical, local ocular (i.e. subconjunctival, intravitreal, retrobulbar, intracameral), and systemic. Each one of these methods has its benefits and its challenges. As such, the pharmaceutical composition comprising the system according to the invention should be adapted to these methods of delivery.

The most appropriate method of administration depends on the area of the eye to be treated. The administration form and the pharmaceutically acceptable medium according to the invention thus also need to be suitable for administration to the area of the eye to be treated.

For example, a system according to the invention may be suitable for subretinal administration. To date, subretinal delivery has been widely applied by scientists and clinicians as a more precise and efficient route of ocular drug delivery for gene therapies and cell therapies including stem cells in diseases such as retinitis pigmentosa.

In particular, subretinal injection has more direct effects on the targeting cells in the subretinal space.

These ocular administration forms are well known to the skilled and the art and are described, for example, in Peng et al. [45].

Ex vivo or In vitro Method for Specifically Editing an Allele Containing the c.166G>A Mutation in NR2E3 in the Genome.

In a third aspect, the invention refers to an ex vivo or in vitro method for specifically editing an allele containing c.166G>A mutation in NR2E3 in the genome of a subject's cell comprising the steps of:
(i) providing to the cell a site-directed genetic engineering system according to the invention; and
(ii) culturing the cell obtained at step (i), wherein the allele containing c.166G>A mutation in NR2E3 has been edited.

In some embodiment, the method specifically silences an allele containing c.166G>A mutation in NR2E3 in the genome of a subject's cell.

Thus, the invention refers to an ex vivo or in vitro method for specifically silencing an allele containing c.166G>A mutation in NR2E3 in the genome of a subject's cell comprising the steps of:
(i) providing to the cell a site-directed genetic engineering system according to the invention; and
(ii) culturing the cell obtained at step (i), wherein the c.166G>A mutation has been silenced.

In some embodiment, the CRISPR protein of the site-directed genetic engineering system according to the invention binds to a locus cognate to the gRNA of the site-directed genetic engineering system according to the invention and generate a double strand break (DSB) in the said cell, and wherein the recombination of said DSB results in silencing the allele containing the c.166G>A mutation in the genome of said cell.

In some embodiment, the recombination of said DSB is non-homologous end joining (NHEJ) recombination

In some embodiment, the method specifically corrects an allele containing c.166G>A mutation in NR2E3 in the genome of a subject's cell.

Thus, the invention refers to an ex vivo or in vitro method for specifically correcting an allele containing c.166G>A mutation in NR2E3 in the genome of a subject's cell comprising the steps of:
(i) providing to the cell a site-directed genetic engineering system according to the invention; and at least one donor nucleic acid that serves as a repair template for the mutated NR2E3 gene, in particular in the form of a single-stranded oligodeoxynucleic acid (ssODN);
(ii) culturing the cell obtained at step (i), wherein the said at least one donor nucleic acid is integrated in the cell genome so as to correct the c.166G>A mutation in NR2E3 gene.

The presence of the at least one donor nucleic acid that serves as a repair template is to direct the cell towards an alternative repair pathway, i.e. towards homology-directed repair (HDR). To accomplish this, the donor nucleic acid that serves as a repair template, in particular in the form of ssODN, bears the desired sequence, which must be introduced in the genome of the cell. A certain number of cells will use this template to repair the broken sequence via homologous recombination, thereby incorporating the desired corrections into the genome.

In some embodiment, the nucleic acid that serves as a repair template bears the non-mutated NR2E3 gene (namely, the NR2E3 gene without the c.166G>A mutation).

In some embodiment, the donor nucleic acid that serves as a repair template may be selected from the group consisting of the sister chromatid in the other allele (i.e non-mutated allele) of the cell, a exogenous plasmid/vector or a single-stranded oligonucleotides (ssODN).

In some embodiment, the donor nucleic acid that serves as a repair template is a single-stranded oligonucleotide (ssODN).

As used ssODNs have been shown to be effective and powerful templates for directing HDR upon DSB in the genome [51]). A previous study demonstrated that asymmetric ssODN complementary to the non-targeted strand enhance HDR.

In some embodiment, the donor nucleic acid that serves as a repair template for the mutated NR2E3 gene are designed using the reference sequence for NR2E3 (Genbank NM_014249 or NM_016346).

In a particular embodiment, the at least one donor nucleic acid that serves as a repair template comprises part of the exon 2 of the NR2E3 gene.

In some embodiment, the subject's cell is induced pluripotent stem cell (iPSC), photoreceptor cell or retinal progenitor cell.

In some embodiment, the subject's cell is photoreceptor cell.

As such, in a particular embodiment, the iPSC, photoreceptor cell or progenitor precursor cell according to the invention is derived from an in vitro processing of a cell previously collected from a subject having an allele containing the c.166G>A mutation in NR2E3 gene.

In some embodiment, the subject have a heterozygous c.166G>A mutation in the NR2E3 gene.

In a particular embodiment, the subject is afflicted with retinitis pigmentosa, in particular with autosomal dominant retinitis pigmentosa, and more particularly with NR2E3-associated autosomal dominant retinitis pigmentosa

iPSCs, photoreceptor cell or retinal progenitor cell as described herein are preferably purified. Many methods for purifying iPSCs or ocular cells, such as photoreceptor cell or retinal progenitor cell, are known in the art. As used herein, “purified iPSCs” or “purified ocular cells” means that the recited cells make up at least 50% of the cells in a purified sample; more preferably at least 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more of the cells in a purified sample.

The cells selection and/or the cells purification can be performed by using both positive and negative selection methods to obtain a substantially pure population of cells.

In one aspect, fluorescence activated cell sorting (FACS), also referred to as flow cytometry, can be used to sort and analyze the different cell populations. Cells having the cellular markers specific for iPSC are tagged with an antibody, or typically a mixture of antibodies, that binds the cellular markers. Each antibody directed to a different marker is conjugated to a detectable molecule, particularly a fluorescent dye that can be distinguished from other fluorescent dyes coupled to other antibodies. A stream of stained cells is passed through a light source that excites the fluorochrome and the emission spectrum from the cells detects the presence of a particular labelled antibody. By concurrent detection of different fluorochromes, cells displaying different sets of cell markers are identified and isolated from other cells in the population. Other FACS parameters, including, by way of example and not limitation, side scatter (SSC), forward scatter (FSC), and vital dye staining (e.g., with propidium iodide) allow selection of cells based on size and viability.

In another aspect, immunomagnetic labelling can be used to sort the different cell population. This method is based on the attachment of small magnetizable particles to cells via antibodies or lectins. When the mixed population of cells is placed in a magnetic field, the cells that have beads attached will be attracted by the magnet and may thus be separated from the unlabeled cells.

In a particular embodiment, the cell previously collected and from which the iPSC, photoreceptor cell or retinal progenitor cell is derived may be an autologous cell, i.e. a cell collected from the subject bearing an allele containing the c.166G>A mutation in the NR2E3 gene, and to which subsequent administration of the cells corrected by the method disclosed herein is contemplated.

“Autologous” refers to deriving from or originating from the same patient or individual. An “autologous transplant” refers to the harvesting and reinfusion or transplant of a subject's own cells or organs. Exclusive or supplemental use of autologous cells can eliminate or reduce many adverse effects of administration of the cells back to the host, particular host reaction.

In another embodiment, the initial population of iPSCs, photoreceptor cell or retinal progenitor cell may be derived from an allogeneic donor or from a plurality of allogeneic donors. The donors may be related or unrelated to each other, and in the transplant setting, related or unrelated to the recipient (or individual).

Genetically Modified Induced Pluripotent Stem Cell and its use for Treating Retinitis Pigmentosa

The present invention also relates to a genetically modified cell obtainable by the method according to the invention as defined above.

In particular, the invention relates to a genetically modified cell wherein the allele containing c the c.166G>A mutation in NR2E3 gene have been silenced, obtainable by a method according to the invention as defined above.

In some embodiment, the genetically modified cell obtainable by the method of the invention is iPSC, photoreceptor cell or retinal progenitor cell.

Treatment based on the administration of photoreceptor cell or retinal progenitor cell obtainable according to the invention may be used in cell therapy for treating a large number of patients afflicted with retinitis pigmentosa, in particular with autosomal dominant retinitis pigmentosa, and more particularly with NR2E3-associated autosomal dominant retinitis pigmentosa.

In order to be used as a medicament, the genetically modified induced pluripotent stem cells (iPSC) according to the invention should be cultured into a particular differentiated cell, such as retinal progenitor cells or retinal organoid.

Treatment based on the administration of cell differentiated from an iPSC obtainable according to the invention may be used in cell therapy for treating a large number of patients afflicted with retinitis pigmentosa, in particular with autosomal dominant retinitis pigmentosa, and more particularly with NR2E3-associated autosomal dominant retinitis pigmentosa.

Accordingly, the invention refers to a retinal organoid differentiated from genetically modified iPSCs according to the invention.

Accordingly, the invention refers to a population of retinal progenitor cells differentiated from genetically modified iPSCs according to the invention.

The population of retinal progenitor cell are obtained after culturing a genetically modified iPSC as prepared according to the invention until it has differentiated into a retinal progenitor cell. The culture and cell differentiation is done under appropriate conditions and includes one or more lineage-specific differentiation factors. These differentiation factors are well known to one skilled in the art and are selected according to the end-cell that is needed.

In some embodiment, the population of retinal progenitor cells differentiated from a genetically modified iPSCs according to the invention is a population of photoreceptor cells, a population of photoreceptor precursor and/or population of rod precursor cell.

The present invention also relates to a pharmaceutical composition comprising a population of the genetically modified cell of the invention and/or the population of retinal progenitor cells differentiated from a genetically modified iPSC according to the invention.

A further object of the invention relates to a population of genetically modified cell according to the invention; a population of retinal progenitor cells differentiated from a genetically modified iPSC according to the invention; or a pharmaceutical composition according to the invention for use as a medicament.

The present invention also relates to the genetically modified cell according to the invention; the population of retinal progenitor cells differentiated from a genetically modified iPSC according to the invention; or the pharmaceutical composition according to the invention for use in therapy.

In some embodiment, the therapy is cell-based therapy or regenerative medicine.

In particular, the invention relates to the genetically modified cell according to the invention; the population of retinal progenitor cells differentiated from a genetically modified iPSC according to the invention; or the pharmaceutical composition according to the invention for use in the treatment of retinitis pigmentosa in subject in need thereof.

In some embodiment, the retinitis pigmentosa is autosomal dominant retinitis pigmentosa, and more particularly NR2E3-associated autosomal dominant retinitis pigmentosa.

In some embodiment, the genetically modified cell according to the invention; the population of retinal progenitor cells differentiated from a genetically modified iPSC according to the invention; or the pharmaceutical composition according to the invention can be used in combination with photoreceptor cell, photoreceptor precursors or rod precursor cell.

In some embodiment, the genetically modified cell according to the invention; the population of retinal progenitor cells differentiated from a genetically modified iPSC according to the invention; or the pharmaceutical composition according to the invention can be used in combination with other agents and compounds that enhance the therapeutic effect of the administered cells.

In another embodiment, the population of retinal progenitor cells differentiated from a genetically modified iPSCs according to the invention can be used in combination with therapeutic compounds that enhance the differentiation of said cells differentiated from a genetically modified iPSC.

These therapeutic compounds have the effect of inducing differentiation and mobilization of the cells that are endogenous, and/or the ones that are administered to the individual as part of the therapy.

The invention will be further illustrated by the following example. However, the example should not be interpreted in any way as limiting the scope of the present invention

### FIGURES

FIG. 1: Characterization of the G56R iPSC line from the NR2E3-adRP patient. A) Bright-field microscopy of a G56R iPSC colonies. B) Sanger sequencing of exon 2 of NR2E3 showing (boxes) a G at cDNA position 166 in the DNA of WT iPSC and the G>A transition at c.166 in the G56R iPSC line. C) RT-PCR detection of the reprogramming vectors in RNA from non-transduced patient fibroblasts (Fibro; negative control), fibroblasts transduced with the Sendai virus vectors (Fibro+SeV; positive control) and G56R iPSC using primers specific for either the vector backbone (SeV) or for each reprogramming cassette: polycistronic KLF4, OCT3/4 and SOX2 (KOS), or monocistronic KLF4 and MYC. Primers for the housekeeping gene GAPDH were used as a positive control for the PCR reaction. D) Karyotype analysis of the G56R iPSC line showing normal chromosomal number and structure. E) qPCR analysis of the expression of the host pluripotency genes NANOG (top), OCT3/4 (middle) and LIN28A (bottom) in cDNA from non-transduced patient fibroblasts (Fibro; negative control), fibroblasts transduced with the Sendai virus vectors (Fibro+SeV) and G56R iPSC. Results are expressed as mean±SEM (n=3).

FIG. 2: Design and efficiency testing of gRNAs. A) Sequence of exon 2 of NR2E3 in the DNA of the NR2E3-adRP patient showing the position of the c.166G>A mutational site (in bold). The position of gRNA1 is indicated by a line and the corresponding NGG PAM sequence is in bold and underlined. The position of gRNA2 is indicated by a line and the corresponding NGA PAM sequence is indicated in bold and underlined. B) Gel electrophoresis of the T7E1 assay in G56R and WT iPSC that were mock-transfected or transfected with gRNA1 or gRNA2. The minus sign indicates the absence, and the plus sign indicates the presence, of T7E1. Molecular weight (MW) marker, 100 bp ladder.

FIG. 3: Generation of G56R-CRISPR iPSC lines. A) Representative electropherogram of the sequence flanking the DSB site in the DNA of the G56R-CRISPR3/4 iPSC clones. The sequence begins at the c.166G>A mutational site. The introduced nucleotide (c.174insA) is boxed. B) Alignment of the Sanger sequencing results for the same region shown in (A) in the wildtype, G56R and four G56R-CRISPR IPSC clones. The mutational site is shown in bold. The indels in are indicated by a dash or underlined. The PAM sequence is shown in bold. C) Graphical representation of the protein structure of 410 aa WT NR2E3 (top) showing the DNA binding domain (DBD), the ligand binding domain (LBD) and the N-terminus and the hinge region. Predicted effect on NR2E3 (bottom) following introduction of the frameshift mutation on the G56R (indicated by a star in the truncated DBD) mutant allele. The thick bar indicates a non-NR2E3 protein sequence prior to the premature termination at 139 aa.

FIG. 4: Characterization of the G56R-CRISPER iPSC lines. A) qPCR analysis of the expression of the host pluripotency genes NANOG (top), OCT3/4 (middle) and LIN28A (bottom) in cDNA from G56R and G56R-CRISPR2/3 iPSC. Results are expressed as mean+SEM (n=3). B) Representative digital qPCR analysis of the G56R-CRISPR3 iPSC line for the presence of the most commonly rearranged chromosomal regions detected in iPSC. A copy number of ˜2 for the autosomes, and 1 for the sex chromosomes, is indicative of stability.

FIG. 5: Effect of mutant allele knockout on NR2E3 expression. Western blot analysis of HEK293 cells mock-transfected or transfected with the pCDNA3 constructs expressing WT, G56R or G56R-CRISPR2 NR2E3 and hybridized with (A) a mouse monoclonal anti-NR2E3 antibody (NR2E3 (m)) or (B) a rabbit polyclonal NR2E3 antibody (NR2E3 (r)). Both anti-NR2E3 antibodies detected a 45-kDa protein in WT and G56R-expressing cells. The mouse monoclonal anti-NR2E3 antibody detected a truncated 16-kDa protein in the G56R-CRISPR2-expressing cells (A). Loading controls, an anti-tubulin antibody detected a 55-kDa protein (A); an anti-actin antibody detected a 42-kDa protein (B). C) Representative images following IF analysis of COS7 cells transfected with the pcDNA3 constructs expressing wild type (top), G56R (middle) or G56R-CRISPR2 (bottom) NR2E3. D) Quantification of the percentage of COS7 cells transfected with the pCDNA3 constructs expressing wild type or G56R NR2E3 showing the mixed perinuclear and nuclear versus mainly nuclear localization profiles. Results are expressed as mean±SEM of 3 independent experiments (n=3;p <0.05; Mann and Whitney test); each experimental value represented the mean of 5 separate fields. E) Western blot analysis following differential centrifugation of cytosolic (left) and nuclear (right) fractions of HEK293 cells mock-transfected (lanes 1) or transfected with the pCDNA3 constructs expressing WT (lanes 2), G56R (lanes 3) or G56R-CRISPR2 (lanes 4) NR2E3. Bands corresponding to 45-kDa NR2E3 were detected in both fractions for WT and G56R-transfected cells, whereas a 16-kDa protein was only detected in the cytosolic fraction of G56R-CRISPR2-transfected cells. To control for fraction purity, both membranes were hybridized with anti-tubulin (55 kDa) and anti-histone H3 (15 kDa). A non-specific 40-kDa band could be detected after incubation with the H3 antibody on both membranes.

### EXAMPLE

Material & Methods

Skin Biopsy and Fibroblast Culture

The skin biopsy of the NR2E3-associated adRP patient volunteer was performed at the National Reference Center for Inherited Sensory Diseases (Maolya) following signed informed consent. The biomedical research study was approved under the authorization number 2014-A00549-38 by the French National Agency for the Safety of Medicines and Health Products (ANSM). The biopsy and emerging fibroblasts were cultured in AmnioMAX C100 basal media with L-glutamine (Gibco, Thermo Fisher Scientific, Villebon sur Yvette, France) containing 10% decomplemented foetal bovine serum (FBS; Gibco), 1% penicillin-streptomycin amphotericin B (Gibco) and 2% AmnioMax-C100 supplement (Gibco). Fibroblasts were passaged using 0.25%Trypsin (Gibco) and cryo-preserved in FBS supplemented with 10% DMSO (Sigma Aldrich, St. Quentin Fallavier, France).

iPSC Reprogramming and Culture

Protocol as previously described [29]. Fibroblasts were seeded in high-glucose DMEM with GlutaMAX (Gibco) containing 10% FCS, 1% non-essential amino acids (NEAA; Gibco) and 55 mM β-mercaptoethanol (Gibco). At day 7 post-transduction, transduced fibroblasts were passaged onto Matrigel-coated plates (Corning hESC-Qualified Matrix, Dominique Dutscher, Brumath, France) and the following day, the medium changed to TeSR-E7 Basal Medium (STEMCELL Technologies, Grenoble, France). Emerging iPSC colonies were mechanically transferred into supplemented Essential (E) 8 Medium (Gibco) and subsequently passaged using Versene Solution (Gibco).

DNA Sequencing

Genomic DNA was isolated from fibroblasts using the DNeasy Blood & Tissue Kit (Qiagen, Courtaboeuf, France) and amplified by PCR using AmpliTaq Gold™ 360 Master Mix (Applied Biosystems, Thermo Fisher) and NR2E3-specific primers (Table S1) on a Veriti thermocycler (Applied Biosystems). The amplicons were cleaned with the ExoSAP-IT PCR Clean-up kit (GE Healthcare, Velizy Villacoublay, France) and sequenced using the BigDye Terminator Cycle Sequencing Ready Reaction kit V3.1 (Applied Biosystems). Analyses were performed on an Applied Biosystems 3130xL genetic analyser.

RT-PCR and qPCR

Total RNA from fibroblasts and iPSC was extracted with the RNeasy Mini Kit (Qiagen). cDNA was synthesized from 500 ng of RNA with the SuperScript III First-Strand Synthesis

System using random hexamers (Life Technologies, Thermo Fisher Scientific). The cDNA was diluted 1:10 and 2 μL used per PCR reaction (10 μl total). The qPCR reaction was performed using the FastStart SYBR Green I Master mix and the LightCycler 480 II thermal cycler (Roche, Meylan, France). Gene expression was normalized to GAPDH expression.

Chromosome Integrity Analyses

Preparation of the G56R iPSC for chromosome spreading was performed as previously described [52]. Twenty metaphase spreads were counted and karyotype analysis was performed by the Chromostem facility (CHU Montpellier, France). Analysis of the G56R-CRISPR iPSC was performed using the iCS-digital TM PSC test by Stem Genomics (Montpellier, France).

Embryoid Body and Retinal Organoid Differentiation

iPSC were differentiated into EBs as previously described, without modification [30]. iPSC were differentiated into retinal organoids based on a previously described protocol [37]. Briefly, iPSC were cultured in E8 on Matrigel dishes to 70-80% confluency and the medium was changed to E6; this was defined as day 0 of differentiation. At day 2, 1% N2 supplement was added. On day 28, neuro-retinal organoids were excised with a scalpel and transferred to ultra-low attachment 24-well plates for individual free-floating culture and were maintained in DMEM/F12, 1:1, medium with Glutamax (Gibco) containing 1% MEM nonessential amino acids (Gibco), 1% Glutamax (Gibco), 1% B27 supplement (Gibco), 10 units/ml Penicillin and 10 μg/ml Streptomycin (Gibco), and supplemented with 10 ng/ml of animal-free recombinant human FGF2 (Miltenyi Biotec, Paris, France). At day 35, FGF2 was removed from the medium and 10% FBS was added. At day 85, B27 supplement was replaced by B27 supplement without vitamin A (Gibco). During the entire differentiation protocol, the medium was changed 3 times per week; for the free-floating culture, half the medium was refreshed 3 times per week.

Immunofluorescence Staining

iPSC and embryoid bodies were fixed in 4% PFA for 20 minutes at room temperature, permeabilized with 0.1% Triton X-100 (Sigma-Aldrich) and blocked with 5% BSA and 10% donkey serum (Millipore, Saint Quentin en Yvelines, France) for 1 h. Retinal organoids were collected at day 180 of retinal maturation, washed twice in PBS, fixed in 4% PFA for 20 min at 4° C., incubated in 20% sucrose overnight at 4° C., embedded in Tissue freezing medium

(Microm Microtech, Brignais, France) and frozen on dry ice. Embedded organoids were cut into 10 μm cryosections using a Leica CM3050 cryostat and collected on Superfrost Plus glass slides (Thermo Scientific). The primary and secondary antibodies are listed in Table S2 and Table S3, respectively. Primary antibodies were incubated overnight at 4° C.; an overnight incubation without primary antibody was used for the negative control. The secondary antibodies and 0.2 μg/ml bisBenzimide (Sigma-Aldrich) were incubated for 1 h at room temperature. Samples were observed using a Zeiss ApoTome 2 Upright wide-field microscope or a Confocal Zeiss LSM700 microscope.

Design of sgRNAs and Plasmid Construction

CCTOP (https://crispr.cos.uni-heidelberg.de), CRISPOR (http://crispor.tefor.net) and MIT CRISPR online design tool (https://zlab.bio/guide-design-resources) were used for designing sgRNA1 for eSpCas9, and CCTOP and CRISPOR for designing sgRNA2 for VQR Cas9. We only retained gRNAs that spanned the mutational site. The plasmids used for cloning the gRNAs were eSpCas9(1.1) (#79145; Addgene) and p458 VQR (#101727, Addgene). The complementary oligonucleotides for each sgRNA were produced by Eurogentec (Angers, France) (Table S1). The oligonucleotide pairs were annealed after 5 min of denaturation at 95° C. and slow-cooled to 50° C. (ramp rate 0.2° C./sec), incubated at 50° C. for 10 min and further cooled to 4° C. (ramp rate 1° C./sec). The corresponding plasmids were digested with the BbsI restriction enzyme prior to ligation using the T4 DNA ligase (Promega, Charbonnieres les Bains, France) overnight at 4° C., according to the manufacturer's instructions.

iPSC Transfection and FACS

iPSC transfection and sorting was performed as previously described [34]. iPSC were cultured in mTeSR1 medium (STEMCELL Technologies, Grenoble, France) pre- and post-transfection. Large-scale and high-purity plasmids were prepared using the Qiagen EndoFree Plasmid Maxi kit. The iPSCs were dissociated with Accutase (STEMCELL Technologies), and 1.5×106 cells were electroporated with 5 μg of plasmid DNA using the Amaxa nucleofector system (Lonza, Levallois-Perret, France). Following transfection, cells were resuspended in mTeSR1 medium supplemented with 10 mM Rho-associated kinase (ROCK) inhibitor Y-27632 (StemMACS; Miltenyi Biotec, Paris, France) and seeded in 24-well plates. Forty-eight hours post-transfection, EGFP-positive cells were single cell-sorted by FACS (FACSAria III, Becton Dickinson, San Jose, CA, USA) into 96-well plates. Two to three weeks post-electroporation, surviving colonies were manually picked and expanded for culture and screening.

T7 Endonuclease I Assay

The T7EI assay was performed in transfected iPSC as described in [34]. The region surrounding the mutation was amplified with specific primers (Table S1) by the high-fidelity TaKaRa polymerase (Thermo Fisher Scientific). The PCR products were then denatured at 95° C. for 5 minutes and gradually reannealed at 95-85° C. (ramp rate of −2° C./sec) followed by 85-25° C. (ramp rate of −0.3° C./sec) to allow the formation of heteroduplexes. The reannealed amplicons were then incubated with T7EI (New England Biolabs, Evry, France) at 37° C. for 1 h and the reaction stopped by the addition of Proteinase K (Qiagen) at 37° C. for 5 min. The digested products were analyzed by 1% ultrapure agarose gel electrophoresis.

Off-target Analysis

The possible off-targets regions were predicted by the CRISPOR software, which used the MIT and CFD scores. The top 5 exonic off-targets for each of the scores (10 in total) were selected for analysis. Primer Blast was used to design primers flanking the predicted regions (Table S1). PCR amplification and sequencing was performed on the genomic DNA isolated from the G56R-CRISPR iPSC lines and a WT iPSC line as described above.

Mutagenesis and NR2E3 cDNA Plasmid Construction

The NR2E3 cDNA was isolated from the Clontech Human Retina QUICK-Clone cDNA pool (TaKaRa Bio Europe, Saint Germain en Laye, France) using specific primers and the amplicon was cloned into the pGEM-T Easy vector system (Promega), according to the manufacturer's instructions. Mutagenesis was performed using the QuickChange Site-Directed

Mutagenesis Kit (Agilent Technologies, Montpellier, France) to introduce the c.166G>A (G56R) and c.173_174delAC (G56R-CRISPR2) mutations. The WT and mutated NR2E3 sequences were verified by Sanger sequencing prior to sub-cloning into the pcDNA3 expression system (Invitrogen; kindly provided by Dr. J. Deveaux, INM). For sub-cloning, the pGEM-T Easy constructs and the pcDNA3 plasmid were both digested by the restriction enzyme EcoRI. The pcDNA3 plasmid was dephosphorylated using the FastAP thermosensitive alkaline phosphatase (Thermo Fisher Scientific), and both the vector and inserts were purified using the NucleoSpin Gel and PCR Clean-up kit (Macherey-Nagel) prior to ligation.

HEK293 and COS7 Culture and Transfection

HEK293 (293T; ATCC CRL-3216) and COS7 (ATCC CRL-1651) cells were cultured in DMEM (Gibco) supplemented with 10% FBS. For IF studies, cells were seeded on poly-lysine coated coverslips in 24-well plates and for western blot analysis in 12-well plates at a density of 9×105 cells/cm2 (HEK293) or 5×105 cells/cm2 (COS-7). For the differential centrifugation studies, both cell lines were seeded in 6-well plates at a density of 6×105 cells/cm2 Twenty-four h post-seeding, cells were transfected using lipofectamine 3000 (Thermo Fisher Scientific) and 500 ng (24-well), 1 μg (12-well) or 2.5 μg (6-well) of plasmid DNA. For IF studies, 48 h post-transfection, cells were fixed in 4% PFA for 30 minutes at 4° C., permeabilized with 0.3% Triton X-100 (Sigma-Aldrich) and blocked with 10% BSA and 10% donkey serum for 1 h. Antibodies were diluted in antibody dilution buffer (0.1% Triton X-100, 10% BSA and 1% donkey serum) and IF performed as described for iPSC.

Western Blot Analyses

HEK293 and COS7 cells were washed in PBS and then lysed in RIPA lysis buffer containing Complete protease inhibitor cocktail (Roche). The cell lysates were centrifuged at 20 000 g for 15 min, and the resulting supernatant was quantified using the BCA protein assay kit. The equivalent of 7.5 μg of protein was mixed with Laemmli sample buffer and 1 μμl of β-mercaptoethanol in a volume of 25 μl. Samples were heated at 95° C. for 5 minutes and then loaded onto an AnyKD precast MiniProtean TGX Stain Free Gel. After electrophoresis, the proteins were transferred to a nitrocellulose membrane using a Trans-Blot TurboTM Transfer Pack and System. Following transfer, the membranes were blocked with 0.5% Tween-PBS in 5% skim milk for 1 h at room temperature and incubated with the primary antibodies; the anti-histone H3 antibody was kindly provided by Dr. R Feil, IGMM) diluted in blocking solution, overnight at 4° C. Membranes were then washed three times for 15 min in 0.5% Tween-PBS and incubated with the secondary antibody in blocking solution for 45 min at room temperature. Following three washes, membranes were dried and visualized using an Odyssey CLx imager (Li-Cor Biotechnology).

Differential Centrifugation

Cell fractions were prepared as previously described and adapted to cells in a 6-well format. Briefly, cells were washed with PBS, harvested by scraping and transferred to a 1.5 ml tube. Cells were pelleted by centrifugation 30 sec on a bench-top centrifuge. The pellet was resuspended in 108 μL ice-cold 0.1% NP-40 in PBS and triturated five times using a 200 μL micropipette, then re-centrifuged for 30 sec. For the cytosolic fraction, 36 μl of the supernatant was collected and mixed with 12 μl of 4× Laemmli sample buffer. For the nuclear fraction, the remaining material was re-centrifuged for 30 sec and the pellet was washed once with 0.1% NP-40 in PBS, resuspended in 110 μl 1× Laemmli sample buffer, and sonicated twice for 5 seconds at level 2 using a Sonicator ultrasonic processor XL (Misonix, Inc, Farmingdale, NY). After addition of β-mercaptoethanol, samples were heated at 95° C. for 5 minutes, loaded onto an AnyKD precast MiniProtean TGX Stain Free Gel and processed, as described above.

Results

G56R Fibroblast Reprogramming to iPSC

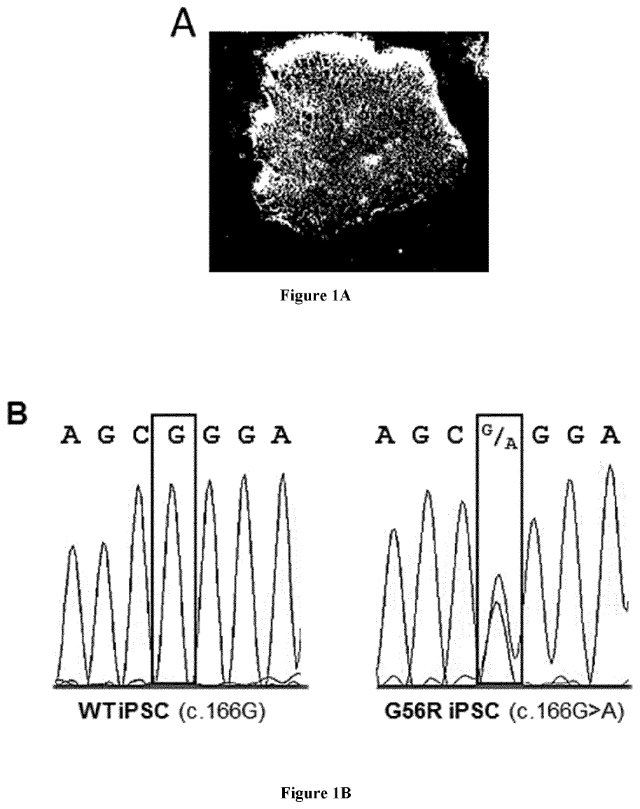

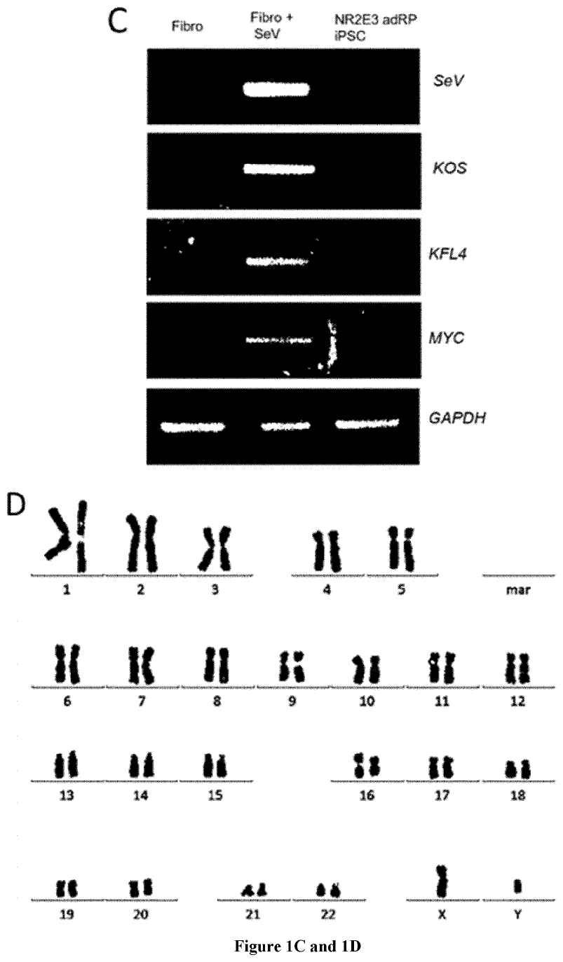

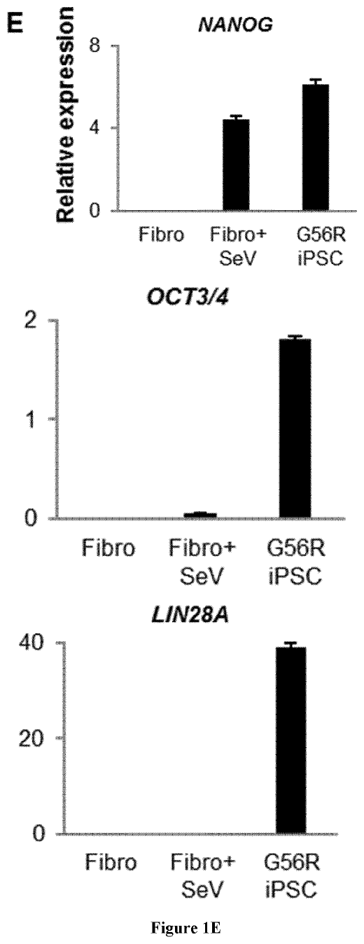

In order to develop a CRISPR/Cas-mediated knockout approach for the G56R mutation, we isolated dermal fibroblasts from a skin biopsy of an NR2E3-associated adRP patient. We reprogrammed the fibroblasts using non-integrative Sendai virus (SeV) vectors carrying Yamanaka's transcription factor cocktail KLF4, OCT3/4, SOX2 and c-MYC. Emerging iPSC showed a typical morphology consisting of tightly packed cells with a distinct border (FIG. 1A). Sanger sequencing confirmed the presence of the c.166G>A mutation in the G56R iPSC as compared to WT iPSC (FIG. 1B). At passage (P) 12, RT-PCR analysis of the exogenous pluripotency genes determined the clearance of the SeV vectors in comparison to non-transduced (negative control) and SeV-transduced (positive control) fibroblasts (FIG. 1C). We observed a normal karyotype of the G56R iPSC at P11, indicative of genetic stability (FIG. 1D). By qPCR analysis, we detected the expression of the host pluripotency genes NANOG, OCT 3/4 and LIN28A in comparison to the lack of expression in non-transduced and SeV-transduced G56R fibroblasts (FIG. 1E); NANOG expression was already detectable in SeV-transduced fibroblasts, consistent with our previous observations [29-30]. Pluripotency was further confirmed by immunofluorescence (IF) staining for NANOG, OCT3/4 and SOX2 (data not shown). In addition, we validated the ability of the G56R iPSC to differentiate into the three germ layers using an embryoid body (EB) differentiation assay and IF staining to detect specific markers: Alpha Fetal Protein (AFP) for endoderm, Smooth Muscle Actin (SMA) for mesoderm and Nestin for ectoderm (data not shown). Taken together, the generated G56R iPSC line passed all the quality controls assessing pluripotency and integrity.

In conclusion, we generated a pluripotent and genetically stable G56R iPSC line from an NR2E3-associated adRP patient, which could be used for therapeutic development.

Designing a G56R-specific Knockout Strategy

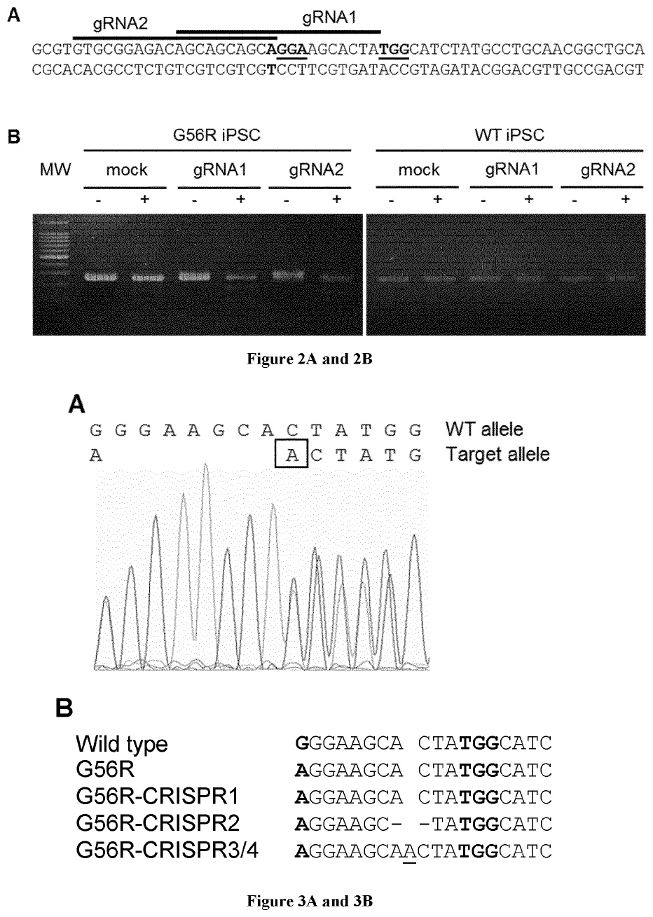

The challenge of a G56R-specific knockout strategy was to design allele-specific gRNAs that would not target the wild type (WT) allele, which could only be achieved by gRNAs that spanned the mutational site. We used three online software for gRNA design: CC TOP (https://crispr.cos.uni-hei-delberg.de), CRISPOR (http://crispor.tefor.net) and MIT CRISPR online design tool (https://zlab.bio/guide-design-resources, no longer available) and identified two overlapping gRNAs that fit our criteria (FIG. 2A). The first, gRNA1, had an NGG PAM sequence, which could be recognized by the SpCas9 as well as by its enhanced specificity variant, eSpCas9, which has reduced off-target effects [31]. The second gRNA, gRNA2, had an NGA PAM sequence, which could be recognized by the engineered VQR variant of SpCas9, created to increase the repertoire of genomic target sequences [32]. When designing gRNA2, we added a mismatched G in its 5′ to enhance transcription driven by the U6 promoter in the expression plasmid We did not include an extra G for gRNA1 that pairs with the eSpCas9 because we previously showed this reduces its activity [34].

We next assessed the ability of the two gRNAs to selectively target the G56R allele. We cloned each gRNA into the corresponding Cas9 plasmid, which contained an enhanced green fluorescent protein (EGFP) tag, and nucleofected WT and G56R iPSCs. Two-days post-transfection, we isolated genomic DNA and performed a T7 endonuclease 1 (T7E1) assay. T7E1 digests DNA when there is mismatch between the two strands, as can occur if there is a frameshift mutation in one of the alleles. A positive digestion result is indicated by multiple bands after gel electrophoresis. As shown in (FIG. 2B), the T7E1 assay was positive for both gRNAs in the patient iPSCs, indicating that at least one of the alleles had been targeted. Two faint bands were also present in the non-transfected patient cells, corresponding to the mismatch due to the heterozygous c.166G>A mutation. However, the T7E1 assay was negative for both gRNAs in the WT iPSCs, indicating that the WT allele was not targeted.

Overall, we identified two gRNAs that specifically targeted the mutant G56R allele. For all subsequent experiments, we retained gRNA1 due to the higher specificity and lower off-target effects of eSpCas9.

Knocking out the G56R Allele in Patient iPSC

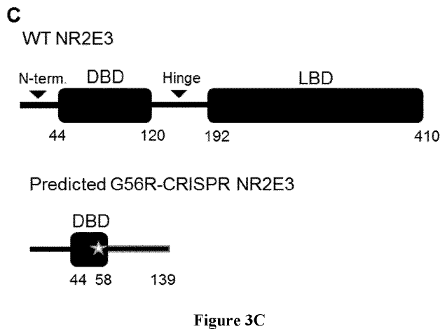

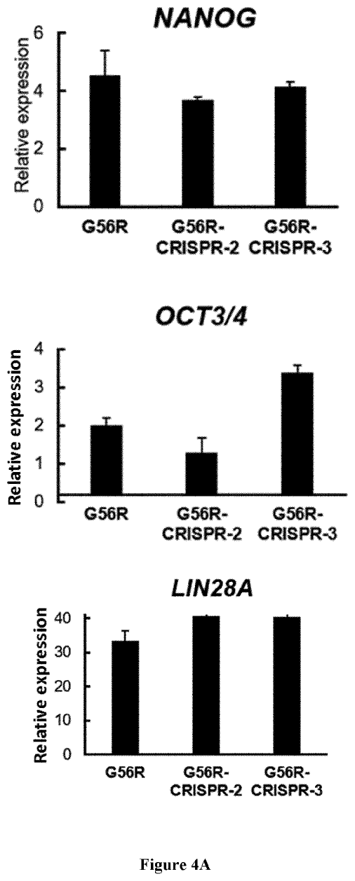

To generate a clonal genome-edited G56R iPSC line, we transfected G56R iPSC with the gRNA1-expressing Cas9 plasmid. The EGFP-positive cells were single cell-sorted by FACS 48-h post-transfection. Two weeks post-sorting, four surviving iPSC colonies emerged. We expanded these clones and Sanger sequenced the mutational site in exon 2 of NR2E3 (FIG. 3A). One clone (G56R-CRISPR1) showed no change following genome-editing, whereas the other three clones (G56R-CRISPR2/3/4) showed the introduction of indels in the mutant allele (FIG. 3B). The mutations were introduced 3-bp upstream of the PAM sequence, at the exact spot where Cas9 is predicted to induce a DSB. The clone G56R-CRISPR2 carried a deletion of two nucleotides (c.173_174delAC) whereas the clones G56R-CRISPR3/4 had the same insertion of one nucleotide (c.174insA). We thus retained only the clones G56R-CRISPR2/3 for further experiments. We verified the sequencing results by individually sub-cloning each allele of the CRISPR clones into the pGEM-T Easy vector system and re-sequencing. We sequenced at least 10 colonies for each clone and identified a 1:1 detection ratio of G56R to WT alleles (data not shown).

A predictive analysis of the protein sequence coded by the G56R-CRISPR2/3 clones identified a frameshift resulting in a PTC, p.(His58Leufs*82) for G56R-CRISPR2 and p.(His58Glnfs*83) for G56R-CRISPR3. It is noteworthy that the new open reading frame was predicted to be identical for both G56R-CRISPR2/3, with the exception that the latter contained one extra aa residue at the point of insertion. According to the predicted protein sequences of the G56R-CRISPR2/3 clones, over half of the DBD (residues 44-120; www.uniprot.org) was removed, as well as the entire LBD (residues 192-410, [35]) (FIG. 3C).

Taken together, the genome-edited G56R-CRISPR clones carried allele-specific indels, which were predicted to disrupt the formation of a full-length protein.

Characterizing the Pluripotency and Stability of G56R-CRISPR iPSC Lines

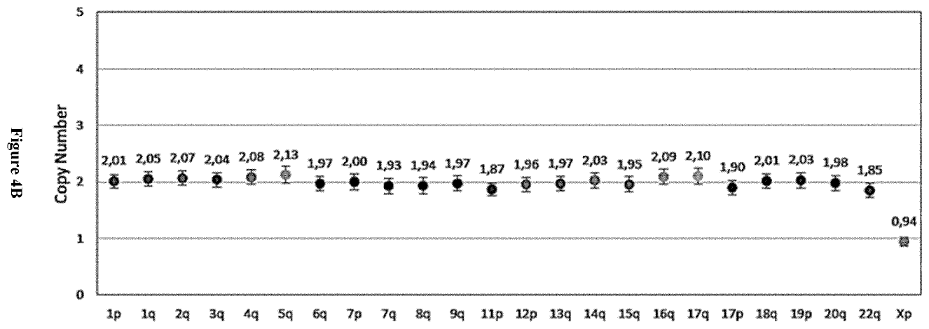

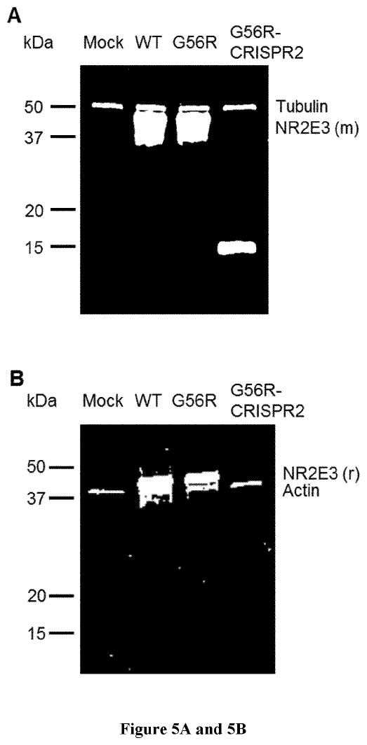

We next assayed whether the G56R-CRISPR2/3 lines retained their iPSC characteristics and genetic stability, in comparison to the parental G56R iPSC line. By qPCR analysis, we detected the expression of the pluripotency genes NANOG, OCT3/4 and LIN28A in the G56R-CRISPR2/3 lines at similar levels to the parental G56R line (FIG. 4A) and, by IF analysis, the expression of the pluripotency markers NANOG, OCT3/4 and SOX2 (data not shown). In addition, the G56R-CRISPR2/3 lines differentiated into the three germ layers, as determined by IF analysis of AFP expression for endoderm, SMA for mesoderm and glial fibrillary acidic protein (GFAP) for ectoderm (data not shown). Moreover, we tested genetic stability of the G56R-CRISPR2/3 iPSC lines by a digital PCR test of the copy number variant (CNV) of the most commonly rearranged chromosomal regions reported in iPSC [36]. A CNV of 2 was detected for the autosomes, and a CNV of 1 for the X chromosome, in both lines indicating that the G56R-CRISPR2/3 iPSCs were genetically stable (FIG. 4B).

Lastly, we evaluated potential off-target events that may have been introduced in the G56R-CRISPR clones. We PCR-amplified the off-target sites for gRNA1 predicted by the CRISPOR software from the DNA of the G56R-CRISPR2/3 lines using specific primers and analyzed the amplicons by Sanger sequencing; to have a complete overview of potential off-target events, we tested in addition G56R-CRISPR4. The sequences for each of these sites in the G56R-CRISPR2/3/4 clones were identical to that of WT, demonstrating the absence of off-target events (data not shown).

In conclusion, following eSpCas9-mediated genome-editing, the resulting G56R-CRISPR iPSC lines retained their pluripotency and genetic stability, and did not harbor off-target events.

Validating Allele-specific Knockout of Mutant G56R NR2E3 at the Protein Level

To validate our allele-specific knockout strategy at the mRNA level, we tested NR2E3 expression levels in iPSC by qPCR. This was, however, inconclusive as firstly, NR2E3 was expressed at very low levels (>30 cycles) and secondly, we were not able to differentiate between the expression of the WT, G56R and G56R-CRISPR2/3 alleles. So, we moved on to directly evaluate the effect of our genome-editing strategy at the protein level using a cell-based overexpression assay. We first amplified WT NR2E3 cDNA from a commercial pool of retinal cDNA by PCR and cloned the amplicon into the pGEM-T Easy vector system. Using this construct as a base, we performed site-directed mutagenesis and generated two additional plasmids, one containing the G56R mutation and the other, the G56R-CRISPR2 frameshift mutation (we did not introduce the G56R-CRISPR3/4 frameshift mutation, as the predicted protein truncation was identical to G56R-CRISPR2). We then sub-cloned the three NR2E3 variants (WT, G56R and G56R-CRISPR2) into the pcDNA3 expression plasmid. The constructs were then transfected separately into HEK293 and COS7 cell lines and we performed western blot and IF studies 48 h post transfection.

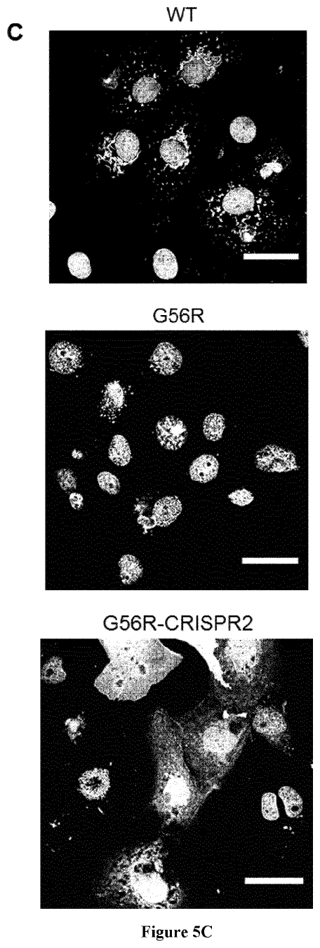

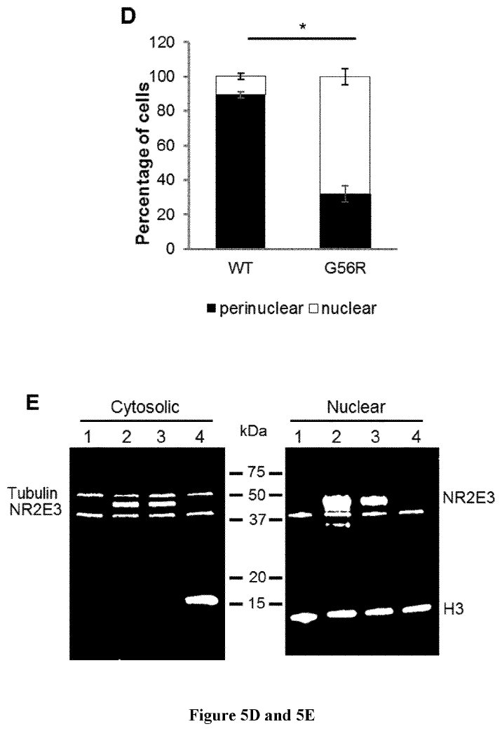

To detect NR2E3 expression by western blot analysis, we tested two antibodies in parallel: a mouse monoclonal antibody (clone H7223) directed to amino acids 2-45 of NR2E3 and a rabbit polyclonal directed to the whole NR2E3 protein. Immunoblotting of the cells transfected with the WT and G56R plasmids detected a full-length 45-kDa NR2E3 protein in both HEK293 (FIG. 5A & 5B) and COS7 (data not shown) cell lines, as expected. By contrast, immunoblotting of the G56R-CRISPR2-expressing cells, only revealed a small protein, that corresponded to the predicted ˜16 kDa size of the truncated NR2E3 (FIG. 5A; 55-kDa tubulin band served as a loading control). Interestingly, the 16-kDa band was only detected by the mouse monoclonal antibody whose epitope is intact in G56R-CRISPR2 and not by the polyclonal antibody (FIG. 5B; a 42-kDa actin band served as a loading control), suggesting that the polyclonal antibody had a higher affinity for the C-terminal part of the protein that was lost in the G56R-CRISPR2 clone.

Following the western blot results, we only used the mouse monoclonal NR2E3 antibody for IF analyses to ensure that we would detect truncated NR2E3 expression. Following transfection of HEK293 (data not shown) and COS7 (FIG. 5C) cells with the three constructs, we detected protein expression in all cases but the cellular localization differed strikingly between the three. WT NR2E3 showed a mixed perinuclear and nuclear localization (FIG. 5C, top) whereas G56R NR2E3 preferentially showed a nuclear localization (FIG. 5C, middle). To evaluate the different localization between WT and G56R we quantified three independent IF experiments. The analysis demonstrated that 89±1.9% of WT-transfected cells showed a perinuclear NR2E3 localization, as compared to 32±4.8% of G56R-transfected cells (FIG. 5D). Conversely, 10.8±2% of WT-transfected cells showed a purely nuclear NR2E3 localization as compared to 68±4.8% of G56R-expressing cells (FIG. 5E). In both cases, these differences were statistically significant (p<0.05).

Furthermore, and in direct contrast to WT and G56R, the expression of the truncated G56R-CRISPR2 NR2E3 protein was much weaker and localized throughout the cytosol (FIG. 5C, bottom). To confirm this localization profile, the cytosolic and nuclear fractions of HEK293 (FIG. 5E) and COS7 (data not shown) cell lines that were transfected with the WT, G56R and G56R-CRISPR2 plasmids were separated by differential centrifugation and analyzed by western blot analysis. Consistent with the (peri)nuclear labelling detected by IF, WT and G56R proteins could be detected in both fractions, whereas the 16-kDa G56R-CRISPR2 protein was exclusively localized to the cytosolic fraction; no band was detected in the nuclear fraction. To verify the purity of the fractions, the membranes were hybridized with an anti-tubulin antibody, as a cytosolic marker, and an anti-histone H3 antibody, as a nuclear marker. A 55-kDa tubulin was only detected in the cytosolic fraction and an ˜15-kDa H3 in the nuclear fraction; a non-specific ˜40-kDa band associated with the anti-H3 antibody was seen in both fractions.

Taken together, overexpression of allele-specific NR2E3 knockout results in a truncated and mis-localized protein in cell lines.

Assessing the Retinal Organoid Differentiation Potential of a G56R-CRISPR iPSC Line

The final part of this study was to investigate whether the genome-edited iPSCs could differentiate into retinal organoids. To this end, we used a previously reported 2D-3D retinal differentiation protocol [37]. Briefly, iPSCs were cultured in 2D feeder-free conditions until neuroepithelial structures with a typical mushroom morphology and a peripheral lamination emerged. At 28 days post-differentiation, we manually excised the organoid structures and individually transferred them to free-floating culture. All three iPSC lines, WT, G56R and G56R-CRISPR2 (data not shown), differentiated into retinal organoids. At 180 days of differentiation, bright-field microscopy showed a characteristic lamination in the organoids that corresponded to the retinal outer nuclear layer (ONL). Furthermore, the retinal organoids presented with a typical brush border, which corresponded to photoreceptor outer segments.

We analyzed the expression of NR2E3 by IF analysis using the mouse monoclonal antibody. NR2E3 was mainly expressed in the ONL of WT, G56R and G56R-CRISPR2 organoids (data not shown). Colocalization studies of NR2E3 and OTX2 expression showed that NR2E3 was restricted to rod nuclei of WT, G56R- and G56R-CRISPR2 organoids, whereas OTX2 also labelled cone nuclei (the OTX2-positive/NR2E3-negative nuclei situated towards the outer rim) (data not shown). Furthermore, as OTX2 is an early developmental marker expressed prior to photoreceptor differentiation, its expression was also detected in an inner layer of nuclei that likely represent retinal progenitor or bipolar cells [38]. Similar results were obtained by colocalization studies of the two partners of NR2E3, CRX and NRL, in the WT, G56R and G56R-CRISPR2 organoids, whereby NRL expression was restricted to the rod nuclei, whereas CRX also labelled cone nuclei (CRX-positive/NRL-negative nuclei in the outer rim) (data not shown).

In conclusion, we showed that allele-specific NR2E3 knockout does not affect the retinal differentiation potential of iPSC nor NR2E3 expression.

Discussion

The G56R mutation in NR2E3 is the second most common mutation causing adRP, a disorder for which there is currently no cure [3]. Genome editing offers a host of therapeutic options for IRDs in terms of gene and cell therapy but the feasibility of clinical translation may be variable. For example, gene correction requires HDR and thus may not reach therapeutic efficiency for gene therapy, due to the post-mitotic nature of photoreceptors, but holds promise for cell therapy. By contrast, a HDR-independent strategy involving specific knockout of a mutant allele could be a potentially efficient gene therapy approach. This would be particularly pertinent in the case of G56R, as this mutation exclusively causes all NR2E3-associated adRP forms [4]. Therefore, a single therapeutic product could potentially treat all patients, rendering it economically attractive. Here, for the first time, we developed a CRISPR/Cas9-mediated allele knockout strategy to treat NR2E3-associated adRP. The efficiency and specificity of this approach suggests that it could be a promising future treatment for NR2E3 patients.

Of the two allele-specific knockout systems that we designed, the most optimal was the one made up of the gRNA1 molecule spanning the G56R mutational site and adjacent to a NGG PAM sequence that can be recognized by eSpCas9. The combination of the allele specificity of the gRNA and the enhanced specificity feature of eSpCas9, resulted in specific targeting of the mutant allele in 75% of the genome-edited iPSC clones; in the remaining 25%, no genome editing events took place. Importantly, the wild type allele was never targeted and no off-target events were detected. The edited clones contained 1- or 2-bp indels within exon 2 of NR2E3, which gave rise to a PTC. It has been established that NMD takes place when a PTC emerges ≥50-55 nt upstream of the last exon-exon junction [40,41]. In our case, the PTCs were located >800 nt upstream of the last exon 7 to exon 8 junction. Therefore, it is highly likely that the mRNA transcribed from the edited DNA would undergo NMD, before producing a mutant protein, corresponding to an effective knockout. In this way, we can exclude the risk that the truncated protein further interferes with the WT NR2E3 allele.

Nevertheless, it has been reported that after CRISPR/Cas-mediated knockout, mRNAs can escape NMD. This can happen through different mechanisms, such as exon skipping, use of alternative initiation codons or conversion of mRNAs with PTC into protein-coding molecules [42]. In addition, sometimes a truncated protein is detectable [43]. To investigate what would happen at the protein level in case the mRNA escaped NMD, we overexpressed the G56R-CRISPR cDNA in an exogenous system wherein NMD would not occur [44]. Under these conditions, we detected a truncated 16-kDa protein from the G56R-CRISPR cDNA, which corresponded to the predicted truncation of NR2E3 within the DBD. Furthermore, this truncated protein showed a defective localization compared to WT NR2E3 and even compared to the mutated G56R NR2E3 protein. More specifically, G56R-CRISPR2 NR2E3 expression was diffused throughout the cytosol, whereas WT NR2E3 expression was restricted to the (peri)nuclear region. Therefore, even if G56R-CRISPR escaped NMD in the photoreceptors, its

altered structure and mislocalization would most likely lead to its degradation [45].

A plausible explanation for the cytoplasmic localization of G56R-CRISPR NR2E3 would be the removal of a nuclear localization signal (NLS). An in silico search of the 410 aa NR2E3 protein sequence (NP_055064.1) for an NLS using the database cNLS mapper [46], identified a putative but low-score bipartite NLS at aa position 69 in the DBD. Consistently, other members of the Nuclear Receptor family are known to contain NLS in the DBD, in the hinge region, or even the LBD [47,48]. The predicted NLS in the NR2E3 DBD is missing from the truncated G56R-CRISPR proteins, which could account for the lack of nuclear targeting. By contrast, the NLS is unaffected by the G56R variant, which is consistent with the nuclear localization of this mutant protein.

What was particularly interesting from this study was the mixed nuclear and perinuclear staining for WT NR2E3, which was a striking contrast from the predominantly nuclear staining of the G56R mutant. These results could suggest that NR2E3 shuttles between the nucleus and the cytoplasm to accomplish its roles in the cell. Different mechanisms have been reported for the export of nuclear receptors including nuclear export signals (NES), protein-protein interactions, and posttranslational modifications [49]. The DBD has been reported to act as a NES for many nuclear receptors, such as the Retinoic X receptor, which belongs to the same subfamily as NR2E3 [49]. Thus, we could hypothesize that this is also the case for NR2E3 and that the G56R mutation, which is in the DBD, could directly affect its nuclear export ability. We also screened the NR2E3 sequence for a potential NES using the LocNES prediction tool [50]. NES were predicted from aa positions 359 to 381 within the LBD, with the highest scoring NES at position 366-380. However, it is difficult to understand how G56R could disrupt this C-terminal signal.

A previous study reported the subcellular localization of a panel of NR2E3 variants,

including G56R [17]. The variants studied were scored according to a nuclear or cytoplasmic localization and, consistent with our data, G56R was reported to be nuclear. Although, there was no strict correlation between the position (DBD versus LBD) of the variants studied and the resulting localization profile, it can be said that the majority of variants resulting in a nuclear localization were situated upstream of aa position 232, and the majority resulting in a cytoplasmic (or mixed) localization were situated downstream (i.e. in the LBD). This could be consistent with the predicted presence of the NES with the LBD of NR2E3 at aa position 366-380. In contrast to our observations, this previous study described a nuclear localization for wild type NR2E3. This is surprising, as there is no doubt as to the perinuclear NR2E3 staining in our study. The cell lines used in the two studies were similar (COS1 versus COST here) so it is unlikely that this underlies the differences observed. However, the previous study used a pcDNA4 His/Max expression system, which fused a 4-kDa Xpress epitope to the NR2E3 protein. It is thus possible that the tag partially interfered with the intracellular trafficking/localization of NR2E3.

To further investigate the effect of allele-specific knockout of G56R NR2E3 in a native environment and, more specifically, on the retinal differentiation potential, we conducted a preliminary iPSC-derived retinal organoid study. We show for the first time that G56R NR2E3 iPSCs can differentiate into retinal organoids. This is consistent with the fact that G56R is associated to progressive RP, which is not a congenital disorder. We also show that G56R-CRISPR iPSCs retain the potential for retinal differentiation. Although, a thorough comparison of the phenotypic differences between WT, G56R and G56R-CRISPR organoids was beyond the scope of this study, we observed that G56R-CRISPR2 organoids expressed NR2E3 in a lamination that was indistinguishable from WT. This suggests that NR2E3 allele-specific ablation would mimic the situation of ESCS loss-of-function mutation carriers who are asymptomatic and that clinical translation of our CRISPR/Cas-mediated knockout strategy would not have negative effects on the human retina.

Taken together, we report here the first proof-of-concept study demonstrating the high specificity and efficiency of allele-specific knockout of the NR2E3 G56R mutant allele using CRISPR/Cas9. This clinically feasible approach represents a promising strategy to treat all patients who develop NR2E3-associated adRP.

### REFERENCES

Throughout this application, various references describe the state of the art to which this invention pertains. The disclosures of these references are hereby incorporated by reference into the present disclosure.

1. Verbakel, S. K et al. Non-syndromic retinitis pigmentosa. Prog. Retin. Eye Res. 30 2018, 66, 157-186.

2. Daiger, S. P et al. Perspective on genes and mutations causing retinitis pigmentosa. Arch. Ophthalmol. 2007, 125, 151-158.

3. Diakatou, M et al. Genome editing as a treatment for the most prevalent causative genes of autosomal dominant retinitis pigmentosa. Int. J. Mol. Sci. 2019, 20.

4. Coppieters, F et al. Recurrent mutation in the first zinc finger of the orphan nuclear receptor NR2E3 causes autosomal dominant retinitis pigmentosa. Am. J. Hum. Genet. 2007, 81, 147-157.

5. Gire, A. I et al. The Gly56Arg mutation in NR2E3 accounts for 1-2% of autosomal dominant retinitis pigmentosa. Mol. Vis. 2007, 13, 1970-5.

6. Blanco-Kelly, F et al. Dominant retinitis pigmentosa, p.Gly56Arg mutation in NR2E3: Phenotype in a large cohort of 24 cases. PLoS One 2016, 11, e0149473.

7. Milam, A. H et al. The nuclear receptor NR2e3 plays a role in human retinal photoreceptor differentiation and degeneration. Proc. Natl. Acad. Sci. U. S. A. 2002, 99, 473-478.

8. Cheng, H et al. Photoreceptor-specific nuclear receptor NR2E3 functions as a transcriptional activator in rod photoreceptors. Hum. Mol. Genet. 2004, 13, 1563-1575.

9. Peng, G. H. et al. The photoreceptor-specific nuclear receptor Nr2e3 interacts with Crx and exerts opposing effects on the transcription of rod versus cone genes. Hum. Mol. Genet. 2005, 14, 747-764.

10. Cheng, H. et al. Photoreceptor-specific nuclear receptor NR2E3 functions as a transcriptional activator in rod photoreceptors. Hum. Mol. Genet. 2004, 13, 1563-1575.

11. Mollema, N. J.; et al. Nuclear receptor Rev-erb alpha (Nrld1) functions in concert with Nr2e3 to regulate transcriptional networks in the retina. PLoS One 2011, 6, e17494.

12. Kobayashi, M. et al. Identification of a photoreceptor cell-specific nuclear receptor. Proc. Natl. Acad. Sci. U. S. A. 1999, 96, 4814-9.

13. Tan, M. H. E. et al. The Crystal Structure of the Orphan Nuclear Receptor NR2E3/PNR Ligand Binding Domain Reveals a Dimeric Auto-Repressed Conformation. PLoS One 2013, 8, 1-13.

14. Aísa-Marín, I. et al. Nr2e3 functional domain ablation by CRISPR-Cas9D10Aidentifies a new isoform and generates retinitis pigmentosa and enhanced S-cone syndrome models. Neurobiol. Dis. 2020, 146, 105122.

15. Escher, P. et al. Mutations in NR2E3 can cause dominant or recessive retinal degenerations in the same family. Hum. Mutat. 2009, 30, 342-351.

16. Roduit, R. et al. Mutations in the DNA-binding domain of NR2E3 affect in vivo dimerization and interaction with CRX. PLoS One 2009, 4, e7379.

17. Kanda, A. et al. A comprehensive analysis of sequence variants and putative disease-causing mutations in photoreceptor-specific nuclear receptor NR2E3. Mol. Vis. 2009, 15, 2174-2184.

18. Doudna, J. A.; Charpentier, E. The new frontier of genome engineering with CRISPR-Cas9. Science. 2014, 346, 1258096-1258096.

19. Wang, H. et al. CRISPR/Cas9 in Genome Editing and Beyond. Annu. Rev. Biochem. 2016, 85, 227-264.

20. Adli, M. The CRISPR tool kit for genome editing and beyond. Nat. Commun. 2018, 9, 1911.

21. Pardo, B. et al. DNA double-strand break repair: How to fix a broken relationship. Cell. Mol. Life Sci. 2009, 66, 1039-1056.

22. Jasin, M.; Rothstein, R. Repair of strand breaks by homologous recombination. Cold Spring Harb. Perspect. Biol. 2013, 5, a012740.

23. Cong, L. et al. Multiplex Genome Engineering Using CRISPR/VCas Systems. Science 2013, 339, 819-823.

24. Christian, M. et al. Targeting DNA double-strand breaks with TAL effector nucleases. Genetics 2010, 186, 756-761.

25. Haider, N. B. et al. Mutation of a nuclear receptor gene, NR2E3, causes enhanced S cone syndrome, a disorder of retinal cell fate. Nat. Genet. 2000, 24, 127-31.

26. Akhmedov, N. B. et al. A deletion in a photoreceptor-specific nuclear receptor mRNA causes retinal degeneration in the rd7 mouse. Proc. Natl. Acad. Sci. U. S. A. 2000, 97, 5551-6.

27. Webber, A. L. et al. Dual role of Nr2e3 in photoreceptor development and maintenance. Exp. Eye Res. 2008, 87, 35-48.

28. Takahashi, K. et al. Induction of Pluripotent Stem Cells from Adult Human Fibroblasts by Defined Factors. Cell 2007, 131, 861-872.

29. Erkilic, N. et al. Generation of a human iPSC line, INMi003-A, with a missense mutation in CRX associated with autosomal dominant cone-rod dystrophy. Stem Cell Res. 2019, 38, 101478.

30. Erkilic, N. et al. Generation of a human iPSC line, INMi004-A, with a point mutation in CRX associated with autosomal dominant Leber congenital amaurosis. Stem Cell Res. 2019, 38, 101476.

31. Slaymaker, I. M. et al. Rationally engineered Cas9 nucleases with improved specificity. Science 2016, 351, 84-8.

32. Kleinstiver, B. P. et al. Broadening the targeting range of Staphylococcus aureus CRISPR-Cas9 by modifying PAM recognition. Nat. Biotechnol. 2015, 33, 1293-1298.

33. Ran, F. A. et al. Genome engineering using the CRISPR-Cas9 system. Nat. Protoc. 2013, 8, 2281-2308.

34. Sanjurjo-Soriano, C. et al. Genome Editing in Patient iPSCs Corrects the Most Prevalent USH2A Mutations and Reveals Intriguing Mutant mRNA Expression Profiles. Mol. Ther.Methods Clin. Dev. 2020, 17, 156-173.

35. Tan, M. H. E. et al. The Crystal Structure of the Orphan Nuclear Receptor NR2E3/PNR Ligand Binding Domain Reveals a Dimeric Auto-Repressed Conformation. PLoS One 2013, 8, e74359.

36. Assou, S. et al. Recurrent Genetic Abnormalities in Human Pluripotent Stem Cells: Definition and Routine Detection in Culture Supernatant by Targeted Droplet Digital PCR. Stem Cell Reports 2020, 14, 1-8.

37. Slembrouck-Brec, A. et al. Reprogramming of adult retinal mtiller glial cells into human-induced pluripotent stem cells as an efficient source of retinal cells. Stem Cells Int. 2019, 2019, 18-21.

38. Chan, C. S. Y. et al. Cell type- And stage-specific expression of Otx2 is regulated by multiple transcription factors and cis-regulatory modules in the retina. Dev. 2020, 147, dev187922.

39. Sanjurjo-Soriano, C.; Kalatzis, V. Guiding Lights in Genome Editing for Inherited Retinal Disorders: Implications for Gene and Cell Therapy. Neural Plast. 2018, 2018, 5056279.

40. Nagy, E.; Maquat, L.E. A rule for termination-codon position within intron-containing genes: when nonsense affects RNA abundance. Trends Biochem. Sci. 1998, 23, 198-199.

41. Popp, M. W. L.; Maquat, L.E. The dharma of nonsense-mediated mRNA decay in mammalian cells. Mol. Cells 2014, 37, 1-8.

42. Tuladhar, R. et al. CRISPR-Cas9-based mutagenesis frequently provokes on-target mRNA misregulation. Nat. Commun. 2019, 10, 1-10.

43. Reber, S. et al. CRISPR-Trap: a clean approach for the generation of gene knockouts and gene replacements in human cells. Mol. Biol. Cell 2018, 29, 75-83.

44. Matsuda, D. et al. Chapter 9 Studying Nonsense-Mediated mRNA Decay in Mammalian Cells; Elsevier Masson SAS, 2008, 449, 177-201.

45. Hegde, R. S.; Zavodszky, E. Recognition and degradation of mislocalized proteins in health and disease. Cold Spring Harb. Perspect. Biol. 2019, 11, a033902.

46. Kosugi, S. et al. Six classes of nuclear localization signals specific to different binding grooves of importina. J. Biol. Chem. 2009, 284, 478-85.

47. Robinson-Rechavi, M. et al. The nuclear receptor superfamily. J. Cell Sci. 2003, 116, 585-586.

48. Porter, B. A. et al. Structure and function of the nuclear receptor superfamily and current targeted therapies of prostate cancer. Cancers (Basel). 2019, 11, 1852.

49. Tecalco-Cruz, A. C. Molecular pathways involved in the transport of nuclear receptors from the nucleus to cytoplasm. J. Steroid Biochem. Mol. Biol. 2018, 178, 36-44.

50. Xu, L. et al. LocNES: a computational tool for locating classical NESs in CRM1 cargo proteins. Bioinformatics. 2015, 31, 1357-65.

51. Strouse, B. et al . (2014). Combinatorial gene editing in mammalian cells using ssODNs and TALENs. Scientific Reports, 4(3791). https://doi.org/10.1038/srep03791.

52. Torriano, S. et al. Pathogenicity of a novel missense variant associated with choroideremia and its impact on gene replacement therapy. Hum. Mol. Genet. 2017, 26, 3573-3584.

1. A site-directed genetic engineering system for specifically editing an allele containing the c.166G>A mutation in NR2E3 in the genome of a subject in need thereof, comprising:
(i) at least one guide nucleic acid comprising the nucleic acid sequence selected from the group consisting of SEQ ID NO: 1, SEQ ID NO:2, and SEQ ID NO:3 and
(ii) at least one Clustered regularly interspaced short palindromic repeats (CRISPR) associated nuclease.

2. The site-directed genetic engineering system according to claim 1, wherein the CRISPR associated nuclease is a CRISPR/Cas nuclease.

3. The site-directed genetic engineering system according to claim 1, wherein the at least one guide nucleic acid and the CRISPR associated nuclease are contained in at least one vectors.

4. The site-directed genetic engineering system according to claim 1, wherein the at least one vector comprises a first vector comprising the at least one guide nucleic acid and a second vector encoding the CRISPR associated nuclease.

5. The site-directed genetic engineering system according to claim 3, wherein the at least one vectors is a viral vector or a non-viral vectors.

6. The site-directed genetic engineering system according to claim 5, wherein the viral vector is selected from the group consisting of retroviral vectors, adenoviral vectors, adeno-associated virus vectors, herpes simplex virus vectors, lentivectors, poxvirus vectors and Epstein-Barr virus vectors.

7. A method of treating retinitis pigmentosa in a subject in need thereof, comprising administering to the eye of the subject a cell that has been edited by the method of claim 1.

8. The method according to claim 7, wherein the retinitis pigmentosa is autosomal dominant retinitis pigmentosa.

9. The method according to claim 7, wherein, the subject has a heterozygous c.166G>A mutation in the NR2E3 gene.

10. An ex vivo or in vitro method for specifically editing an allele containing a c.166G>A mutation in the NR2E3 gene in the genome of a subject's cell, comprising the steps of:
(i) providing to the subject's cell a site-directed genetic engineering system according to claim 1; and
(ii) culturing a cell obtained at step (i), wherein an allele containing c.166G>A mutation in NR2E3 has been specifically edited in the cell.

11. The method according to claim 10, wherein the subject's cell is an induced pluripotent stem cell (iPSC), a photoreceptor cell or a retinal progenitor cell.

12. A genetically modified cell obtained by the method according to claim 10.

13. A pharmaceutical composition comprising a plurality of the genetically modified cell of claim 12.

14. (canceled)

15. (canceled)

16. A method for treating retinitis pigmentosa in a subject in need thereof comprising administering to the subject a therapeutically effective amount of the site-directed genetic engineering system of claim 1.

17. The site-directed genetic engineering system according to claim 2, wherein the CRISPR associated nuclease is derived from a Cas9 protein.

18. The site-directed genetic engineering system according to claim 6, wherein the viral vector is an adeno-associated virus vector.

19. The method of claim 8, wherein the autosomal dominant retinitis pigmentosa is NR2E3-associated autosomal dominant retinitis pigmentosa.

20. The pharmaceutical composition of claim 13, wherein the genetically modified cell is a retinal progenitor cell differentiated from a genetically modified iPSC.


SEQUENCE LISTING (11 sequences)

SEQ ID NO: 1 - DNA, 20 bp, Artificial Sequence
  Synthetic gRNA 1
agcagcagcaggaagcacta

SEQ ID NO: 2 - DNA, 20 bp, Artificial Sequence
  Synthetic gRNA 2
gtgcggagacagcagcagca

SEQ ID NO: 3 - DNA, 21 bp, Artificial Sequence
  Synthetic gRNA 1′
gagcagcagcaggaagcacta

SEQ ID NO: 4 - DNA, 60 bp, Homo sapiens
gcgtgtgcggagacagcagcagcaggaagcactatggcatctatgcctgcaacggctgca

SEQ ID NO: 5 - DNA, 60 bp, Homo sapiens
cgcacacgcctctgtcgtcgtcgtccttcgtgataccgtagatacggacgttgccgacgt

SEQ ID NO: 6 - DNA, 18 bp, Homo sapiens
gggaagcactatggcatc

SEQ ID NO: 7 - DNA, 18 bp, Homo sapiens
aggaagcactatggcatc

SEQ ID NO: 8 - DNA, 18 bp, Homo sapiens
aggaagcactatggcatc

SEQ ID NO: 9 - DNA, 16 bp, Homo sapiens
aggaagctatggcatc

SEQ ID NO: 10 - DNA, 19 bp, Homo sapiens
aggaagcaactatggcatc

SEQ ID NO: 11 - DNA, 14 bp, Homo sapiens
gggaagcactatgg